(19) United States
(12) Patent Application Publication
(10) Pub. No.: US 2011/0313415 A1
(43) Pub. Date: Dec. 22, 2011

(54) Medical Devices, Apparatuses, Systems, and Methods

(75) Inventors: Raul Fernandez, Arlington, TX (US); Daniel J. Scott, Dallas, TX (US); Shou Jiang Tang, Ridgeland, MS (US); Jeffrey A. Cadeddu, Dallas, TX (US); Richard A. Bergs, Grand Prairie, TX (US)

(73) Assignee: The Board of Regents of the University of Texas System, Austin, TX (US)

(21) Appl. No.: 13/128,847

(22) PCT Filed: Nov. 11, 2009
(86) PCT No.: PCT/US09/63987

§ 371 Date: Sep. 2, 2011

Related U.S. Application Data
(60) Provisional application No. 61/113,495, filed on Nov. 11, 2008.
(60) Provisional application No. 61/145,469, filed on Jan. 16, 2009.

(51) Int. Cl. A61B 18/14

(52) U.S. Cl. 606/41

(57) ABSTRACT

Apparatuses and systems for enabling electrical communication with a device positionable within a body cavity of a patient. Apparatuses and systems for magnetically positioning a device within a body cavity of a patient. Medical devices. Methods of use.

### CROSS REFERENCE TO RELATED APPLICATIONS

This application claims priority to U.S. Provisional Patent Application Ser. No. 61/113,495, filed on Nov. 11, 2008, and U.S. Provisional Patent Application Ser. No. 61/145,469, filed on Jan. 16, 2009, the entire contents of both of which are incorporated by reference.

### BACKGROUND OF THE INVENTION

1. Field of the Invention

The present invention relates generally to medical devices, apparatuses, systems, and methods, and, more particularly, but not by way of limitation, to medical devices, apparatuses, systems, and methods for performing medical procedures at least partially within a body cavity of a patient.

2. Description of Related Art

For illustration, but without limiting the scope of the invention, the background is described with respect to medical procedures (e.g., surgical procedurals), which can include laparoscopy, transmural surgery, and endoluminal surgery, including, for example, natural orifice transluminal endoscopic surgery (NOTES), single-incision laparosopic surgery (SILS), and single-port laparoscopy (SLP).

Compared with open surgery, laparoscopy can result in significantly less pain, faster convalescence and less morbidity. NOTES, which can be an even less-invasive surgical approach, may achieve similar results. However, issues such as eye-hand dissociation, a two-dimensional field-of-view, instrumentation with limited degrees of freedom, and demanding dexterity requirements can pose challenges for many laparoscopic and endoscopic procedures. One limitation of laparoscopy can be the fixed working envelope surrounding each trocar. As a result, multiple ports may be used to accommodate changes in position of the instruments or laparoscope, for example, to improve visibility and efficiency. However, the placement of additional working ports may contribute to post-operative pain and increases risks, such as additional bleeding and adjacent organ damage.

The following published patent applications include information that may be useful in understanding the present medical devices, apparatuses, systems, and methods, and each is incorporated by reference in its entirety: (1) U.S. patent application Ser. No. 10/024,636, filed Dec. 14, 2001, and published as Pub. No. US 2003/0114731; (2) U.S. patent application Ser. No. 10/999,396, filed Nov. 30, 2004, and published as Pub. No. US 2005/0165449; (3) U.S. patent application Ser. No. 11/741,731, filed Apr. 28, 2007, and published as Pub. No. US 2007/0255273; (4) U.S. patent application Ser. No. 11/833,729, filed Aug. 3, 2007, and published as Pub. No. US 2007/0276424; and (5) U.S. patent application Ser. No. 11/711,541, filed Feb. 27, 2007, and published as Pub. No. US 2008/0208220.

### SUMMARY OF THE INVENTION

Some embodiments include an apparatus for enabling electrical communication with a device positionable within a body cavity of a patient. The device can have an opening and a conductive portion, and the apparatus can comprise: an anchor; and a conductor connected to the anchor; where the anchor and at least a portion of the conductor are insertable (and/or configured to be inserted) through a puncture in an exterior surface of the patient, into the body cavity of the patient, and into the opening of the device; and where the anchor can contact (and/or is configured to contact) the device so as to prevent the anchor and a portion of the conductor from being removed from the body cavity while enabling electrical communication between the conductor and the conductive portion of the device. In some embodiments, the conductor comprises a conductive portion and a layer of insulating material disposed about the conductive portion of the conductor. In some embodiments, the conductor further comprises a second conductive portion disposed about the layer of insulating material, and a second layer of insulating material disposed about the second conductive portion of the conductor.

Some embodiments include a system for enabling electrical communication with a device positionable within a body cavity of a patient. The device can have an opening and a conductive portion, and the system can comprise: a device configured to be positioned within a body cavity of a patient, the device having an opening and a conductive portion; and an apparatus for enabling electrical communication with the device. The apparatus can comprise: an anchor; and a conductor connected to the anchor; where the anchor and at least a portion of the conductor are insertable through a puncture in an exterior surface of the patient, into the body cavity of the patient, and into the opening of the device; and where the anchor can contact the device so as to prevent the anchor and a portion of the conductor from being removed from the body cavity while enabling electrical communication between the conductor and the conductive portion of the device. In some embodiments, the device comprises a light emitting diode (LED), and when electrical communication is enabled between the conductor and the device, electrical communication is enabled between the conductor and the LED.

In some embodiments, the anchor comprises an elongated piece of metallic material. In some embodiments, the anchor fits within a volume that is less than about 1 cubic inch. In some embodiments, the volume of the anchor is defined by a length, width, and a height, and where the length is less than about 1 inch, the width is less than about 0.3 inches, and the height is less than about 0.3 inches. In some embodiments, the opening of the device is at least a portion of a recess that extends into the device. In some embodiments, the opening of the device is at least a portion of a passageway extending through the device. In some embodiments, the conductive portion of the device is adjacent to the opening. In some embodiments, the conductive portion of the device substantially surrounds the opening. In some embodiments, the conductor comprises a first conductive portion and a layer of insulating material disposed about the first conductive portion of the conductor. In some embodiments, the conductor further comprises a second conductive portion disposed about the layer of insulating material, and a second layer of insulating material disposed about the second conductive portion of the conductor.

In some embodiments, when the anchor contacts the device at least one of the anchor and the conductor can contact the conductive portion of the device. In some embodiments, when the anchor contacts the device both the conductor and the anchor can contact the conductive portion of the device. In some embodiments, when the anchor contacts the device the conductor can contact the conductive portion of the device. In some embodiments, when the anchor contacts the device the anchor can contact the conductive portion of the device. In some embodiments, when the anchor contacts the device only a portion of the anchor can contact the conductive portion of the device, and where a portion of the anchor that cannot contact the conductive portion of the device is electrically insulated from the conductive portion of the device.

Some embodiments include an apparatus for magnetically positioning a device within a body cavity of a patient. The apparatus can comprise: a magnetic assembly having a coupling end, the magnet assembly comprising: a primary magnetic field source; a plurality of peripheral magnetic field sources disposed about the primary magnetic field source; and a housing supporting the magnetic assembly; where the volume of the housing and magnetic assembly is less than about 64 cubic inches. In some embodiments, the primary magnetic field source of the magnetic assembly has an N pole and an S pole; each peripheral magnetic field source of the magnetic assembly has an N pole and an S pole; and each magnetic assembly is configured such that the N poles of the peripheral magnetic field sources are adjacent to the S pole of the primary magnetic field source.

Some embodiments of the present apparatuses can comprise two of the magnetic assemblies, where the housing supports the two magnetic assemblies in fixed relation such that their coupling ends are substantially coplanar. In some embodiments comprising two magnetic field source, the primary magnetic field source of each magnetic assembly has an N pole and an S pole, and the S pole of the primary magnetic field source of one magnetic assembly is adjacent that magnetic assembly's coupling end, and the N pole of the primary magnetic field source of the other magnetic assembly is adjacent the other magnetic assembly's coupling end. In some embodiments, the volume of the housing and the magnetic assemblies is less than about 32 cubic inches. In some embodiments, the volume of the housing and the magnetic assemblies is less than about 22 cubic inches.

Some embodiments include an apparatus for magnetically positioning a device within a body cavity of a patient. The apparatus can comprise: two magnetic field sources each having a coupling end; and a housing supporting the two magnetic field sources in fixed relation to one another such that the coupling ends of the two magnetic field sources are adjacent to one another; where the apparatus has a coupling area less than about 8 square inches. In some embodiments, at least one of the two magnetic field sources can have a magnetic assembly comprising: a primary magnet; and a plurality of peripheral magnets disposed about the primary magnet. In some embodiments, the coupling area of the apparatus is less than about 4 square inches. In some embodiments, each magnetic field source has an N pole and an S pole, and where the coupling end of one magnetic field source has the S pole, and the coupling end of the other magnetic field source has the N pole. In some embodiments, the primary magnet of the magnetic assembly has an N pole and an S pole; each peripheral magnet of the magnetic assembly has an N pole and an S pole; and each magnetic assembly is configured such that the N poles of the peripheral magnets are adjacent to the S pole of the primary magnet. In some embodiments, each of the two magnetic field sources has a magnetic assembly comprising: a primary magnet; and a plurality of peripheral magnets disposed about the primary magnetic field source. In some embodiments where each magnetic field source has a magnetic assembly, the primary magnet of each magnetic assembly has an N pole and an S pole; each peripheral magnet of each magnetic assembly has an N pole and an S pole; and each magnetic assembly is configured such that the N poles of the peripheral magnets are adjacent to the S pole of the primary magnet.

Some embodiments include a system comprising: a device comprising a magnetically-attractive material; and an apparatus for moving the device within a body cavity of a patient when the apparatus is outside the body cavity; where the magnetic assembly is magnetically couplable with the magnetically-attractive material of the device through an external surface of the body of the patient such that the device can be moved inside the body cavity by moving the apparatus outside the body cavity. In some embodiments, the apparatus comprises: a magnetic assembly having a coupling end, the magnetic assembly comprising: a primary magnetic field source; and a plurality of peripheral magnetic field sources disposed about the primary magnetic field source. In some embodiments, the magnetically-attractive material of the device comprises a magnet. In some embodiments, the apparatus comprises two of the magnetic assemblies. In some embodiments, the primary magnetic field source of each magnetic assembly has an N pole and an S pole, and the S pole of the primary magnetic field source of one magnetic assembly is adjacent that magnetic assembly's coupling end, and the N pole of the primary magnetic field source of the other magnetic assembly is adjacent the other magnetic assembly's coupling end. In some embodiments, the device has a coupling side, the magnetically-attractive material of the device comprises two magnets that each has an S pole and an N pole, the N pole of one magnet is adjacent the coupling side of the device, and the S pole of the other magnet is adjacent the coupling side of the device.

Some embodiments include a system comprising: a device comprising a magnetically-attractive material; and an apparatus for moving the device within a body cavity of a patient when the apparatus is outside the body cavity. In some embodiments, the apparatus comprises: a magnetic assembly comprising: a primary magnetic field source; and a plurality of peripheral magnetic field sources disposed about the primary magnetic field source; where the magnetic assembly is magnetically couplable with the magnetically-attractive material of the device through an external surface of the body of the patient such that the device can be moved inside the body cavity by moving the apparatus outside the body cavity; and where when the magnetic assembly is magnetically coupled with the magnetically-attractive material of the device at a distance of about 10 millimeters, there is a magnetic attractive force of at least about 2000 grams. In some embodiments, at a distance of about 10 millimeters the magnetic attractive force is at least about 2500 grams. In some embodiments, at a distance of about 10 millimeters the magnetic attractive force is at least about 3000. In some embodiments, at a distance of about 10 millimeters the magnetic attractive force is at least about 3000. In some embodiments, the magnetically-attractive material of the device comprises a magnet. In some embodiments, the apparatus comprises two of the magnetic assemblies.

In some embodiments, the primary magnetic field source of each magnetic assembly has an N pole and an S pole, and the S pole of the primary magnetic field source of one magnetic assembly is adjacent that magnetic assembly's coupling end, and the N pole of the primary magnetic field source of the other magnetic assembly is adjacent the other magnetic assembly's coupling end. In some embodiments, the device has a coupling side, where the magnetically-attractive material of the device comprises two magnets that each has an S pole and an N pole, and where the N pole of one magnet is adjacent the coupling side of the device and the S pole of the other magnet is adjacent the coupling side of the device.

Some embodiments include a system comprising: a device comprising a magnetically-attractive material; an apparatus for moving the device within a body cavity of a patient when the apparatus is outside the body cavity; and an apparatus for enabling electrical communication with the device. The apparatus for moving the device can comprise: a magnetic assembly comprising: a primary magnetic field source; and a plurality of peripheral magnetic field sources disposed about the primary magnetic field source; where the one or more magnetic assemblies are configured to magnetically couple with the magnetically-attractive material of the device through an external surface of the body of the patient such that the device can be moved inside the body cavity by moving the apparatus outside the body cavity. The apparatus for enabling electrical communication with the device can comprise: an anchor; and a conductor connected to the anchor; where the anchor and at least a portion of the conductor are insertable through a puncture in an exterior surface of the patient, into the body cavity of the patient, and into the opening of the device; and where the anchor can contact the device so as to prevent the anchor and a portion of the conductor from being removed from the body cavity while enabling electrical communication between the conductor and the conductive portion of the device.

Some embodiments can include a medical device comprising: a platform at least partially defined by a length and a maximum transverse perimeter, the platform having a longitudinal recess that has a length defined along at least a portion of the length of the platform; an arm having a proximal end, a distal end, and a length extending from the proximal end to the distal end, the arm coupled to the platform such that the arm is movable between (1) a collapsed position in which along the length of the recess the arm is disposed within the maximum transverse perimeter of the platform and (2) an expanded position in which the distal end of the arm is spaced apart from the platform; and a cautery tool coupled to the arm.

Some embodiments include a medical device comprising: a platform at least partially defined by a length and a maximum transverse perimeter, the platform having a longitudinal recess that has a length defined along at least a portion of the length of the platform; an arm having a proximal end, a distal end, a length extending from the proximal end to the distal end, and a longitudinal axis parallel to the length of the arm, the arm coupled to the platform such that the arm is movable between (1) an expanded position in which the distal end is spaced apart from the platform and (2) a collapsed position in which the distal end of the arm is closer to the platform than when the arm is in the expanded position; and a cautery tool coupled to the arm and having a central axis parallel to the longitudinal axis of the arm; where when the arm is in the collapsed position, the central axis of the cautery tool is disposed within the maximum transverse perimeter of the platform.

Some embodiments include a medical device comprising: a platform; an arm coupled to the platform with a pin slidably disposed within a cam slot defined within one of the platform and the arm, the pin being coupled to the other of the platform and the arm, the arm movable between an expanded position and a collapsed position; and a cautery tool coupled to the arm. In some embodiments, the arm is coupled to the platform with two or more pins slidably disposed within first and second cam slots, the first and second cam slots defined within the platform, and the two or more pins supported by and in fixed relation to the arm.

Some embodiments include a medical device comprising: a platform having a proximal end, a distal end, and a length extending between the proximal end and the distal end; an arm having an arm proximal end, an arm distal end, and an arm length extending from the arm proximal end to the arm distal end, the arm coupled to the platform such that the arm is movable between (1) an expanded position in which the arm distal end is spaced apart from the platform and (2) a collapsed position in which the arm distal end is closer to the platform than when the arm is in the expanded position; a cautery tool coupled to the arm, the cautery tool having a tool proximal end, a tool distal end, and a longitudinal tool axis; and a cylinder coupled to the arm and configured to be coupled to a fluid source; where the medical device is configured such that when the cylinder is coupled to a fluid source and actuated, the cautery tool is movable between a non-extended position and an extended position along the longitudinal tool axis.

In some embodiments of the various medical devices, the platform comprises a magnetically-attractive material. In some embodiments, the magnetically-attractive material includes a magnet. In some embodiments, the magnetically-attractive material includes two magnets. In some embodiments, the platform has a coupling side; each magnet has an N pole and an S pole; and the N pole of one magnet is oriented toward the coupling side, and the S pole of the other magnet is oriented toward the coupling side. In some embodiments, the maximum transverse perimeter is less than about 7 inches. In some embodiments, the area circumscribed by the maximum transverse perimeter is less than about 3.2 square inches.

Some embodiments of the present methods include receiving a signal from one or more sensors indicating that a force limit (e.g., a minimum or maximum) has been reached between a device comprising magnetically-attractive material and an apparatus for moving the device within a body cavity of a patient when the apparatus is outside the body cavity; and adjusting the position of a plurality of peripheral magnetic field sources relative to a primary magnetic field source about which they are disposed (either manually or automatically) to alter. One or both of the device and the apparatus may be configured (e.g., with a light source or the like) to visually indicate to an operator that the force limit is reached. Such methods may be used in practice and in actual surgery.

Some embodiments of the present medical devices comprise: a platform having a proximal end, a distal end, and a length extending between the proximal end and the distal end; where the platform comprises a first magnetically attractive member including an upper section having a transverse dimension, and a lower section having a transverse dimension that is larger than the transverse dimension of the upper section. In some embodiments, the platform further comprises a second magnetically attractive member. In some embodiments, the second magnetically attractive member includes an upper section having a transverse dimension, and a lower section having a transverse dimension that is larger than the transverse dimension of the upper section. In some embodiments, the magnetically-attractive members each comprise a magnet. In some embodiments, each magnetically-attractive member comprises a plurality of magnets. In some embodiments, the platform has a coupling side; each magnet has an N pole and an S pole; and the N pole of one magnet is oriented toward the coupling side, and the S pole of the other magnet is oriented toward the coupling side. In some embodiments, the upper section of each magnetically attractive member is adjacent the coupling side of the platform.

Any embodiment of any of the present systems, apparatuses, devices, and methods can consist of or consist essentially of—rather than comprise/include/contain/have—any of the described elements and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1 depicts a graphical representation of one of the present medical devices positioned within a body cavity of a patient and magnetically coupled to a positioning apparatus that is located outside the cavity.

FIG. 2 is an end view of the medical device and positioning apparatus shown in FIG. 1.

FIGS. 3A and 3B are bottom and side-cross-sectional views, respectively, of one of the present positioning apparatuses.

FIG. 4 is a side view of a cylindrical magnet shown with field lines conceptually illustrating its magnetic field.

FIGS. 5A and 5B are perspective views of some of the present magnetic assemblies.

FIG. 6 is a side view of one of the present magnetic assemblies shown with field lines conceptually illustrating the magnetic field of the magnetic assembly.

FIG. 7 is a perspective view of another of the present magnet assemblies.

FIG. 8 is a perspective view of one of the present medical devices.

FIG. 9 is a cross-sectional view of the medical device shown in FIG. 8, taken along line 9-9 in FIG. 8.

FIGS. 10 and 11 are perspective views of different embodiments of the present medical devices.

FIG. 12 is a graphical side view of one of the present positioning devices coupled to one of the present medical devices across tissue.

FIGS. 13A-13G are various views of one of the present medical devices that includes one of the present cautery tools.

FIGS. 13H and 13I are side and cross-sectional views of another embodiment of one of the present medical devices that includes one of the present cautery tools.

FIGS. 14A-14C are various views of another one of the present medical devices that includes one of the present cautery tools.

FIGS. 15A-15D are various views of another one of the present medical devices that includes one of the present cautery tools.

FIGS. 16A-16D are various views, respectively, of another one of the present medical devices that includes one of the present cautery tools.

FIGS. 17A-17C are various views of another one of the present medical devices that includes one of the present cautery tools.

FIGS. 18A-18C are various views of another one of the present medical devices that includes one of the present cautery tools.

FIGS. 19A-19C are various views of another one of the present medical devices that includes one of the present cautery tools.

FIG. 20 is a side view of one of the present systems for enabling electrical communication with a medical device, where the medical device is positioned in a body cavity of a patient.

FIGS. 21A-21D are various views of one of the present medical devices that is adapted for use with one of the present systems for enabling electrical communication with the device.

FIGS. 22A-22E are various views of another embodiment of the present medical devices that is adapted for use with one of the present systems for enabling electrical communication with the device.

FIG. 23A is a perspective view of one of the present apparatuses for enabling electrical communication with one of the present medical devices.

FIGS. 23B-23C are cross-sectional views of a conductor for use with the apparatus shown in FIG. 23A.

FIG. 24 is a perspective view of a deployment needle to which the apparatus shown in FIG. 23A is coupled.

FIG. 25 is a cross-sectional view of the deployment needle and coupled apparatus shown in FIG. 24 taken along line 25-25 in FIG. 24.

FIGS. 26 and 27 are cross-sectional views of the deployment needle and coupled apparatus shown in FIG. 23A at different stages of deployment of the anchor of the apparatus.

FIGS. 28A-28G are different views in a series showing how one of the present medical devices can be coupled to one of the present apparatuses in order to enable electrical communication between the medical device and a power source (not shown).

FIGS. 29A-29C are various views of external locks for use with embodiments of the present systems.

FIGS. 30A and 30B are cross-sectional views of medical devices illustrating alternate embodiments of magnets for some embodiments of the present medical devices.

### DESCRIPTION OF ILLUSTRATIVE EMBODIMENTS

The term “coupled” is defined as connected, although not necessarily directly, and not necessarily mechanically; two items that are “coupled” may be integral with each other. The terms “a” and “an” are defined as one or more unless this disclosure explicitly requires otherwise. The terms “substantially,” “approximately,” and “about” are defined as being largely but not necessarily wholly what is specified, as understood by a person of ordinary skill in the art.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, medical device, or apparatus that “comprises,” “has,” “includes” or “contains” one or more elements possesses those one or more elements, but is not limited to possessing only those one or more elements. Likewise, an element of a system, medical device, or apparatus that “comprises,” “has,” “includes” or “contains” one or more features possesses those one or more features, but is not limited to possessing only those one or more features. For example, a medical device that comprises a platform and a magnetically-attractive material includes the specified features but is not limited to having only those features. Such a medical device could also include, for example, an arm coupled to the platform.

Further, a device or structure that is configured in a certain way is configured in at least that way, but it can also be configured in other ways than those specifically described.

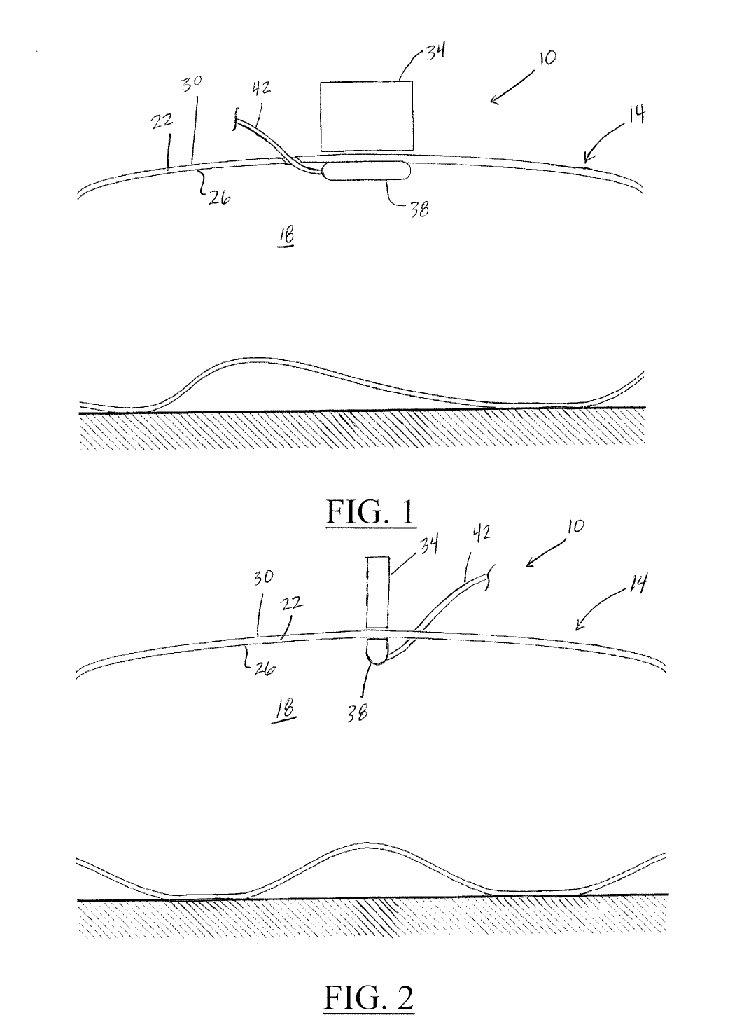

Referring now to the drawings, shown in FIGS. 1 and 2 by reference numeral 10 is one embodiment of a system for medical procedures that can be used with the present invention. System 10 is shown in conjunction with a patient 14, and more particularly in FIG. 1 is shown relative to a longitudinal cross-sectional view of the ventral cavity 18 of a human patient 14, and in FIG. 2 is shown relative to a transverse cross-sectional view of the ventral cavity of the patient. For brevity, cavity 18 is shown in simplified conceptual form without organs and the like. Cavity 18 is at least partially defined by wall 22, such as the abdominal wall, that includes an interior surface 26 and an exterior surface 30. The exterior surface 30 of wall 22 can also be an exterior surface 30 of the patient 14. Although patient 14 is shown as human in FIGS. 1 and 2, various embodiments of the present invention (including the version of system 10 shown in FIGS. 1 and 2) can also be used with other animals, such as in veterinary medical procedures.

Further, although system 10 is depicted relative to ventral cavity 18, system 10 and various other embodiments of the present invention can be utilized in other body cavities of a patient, human or animal, such as, for example, the thoracic cavity, the abdominopelvic cavity, the abdominal cavity, the pelvic cavity, and other cavities (e.g., lumens of organs such as the stomach, colon, or bladder of a patient). In some embodiments of the present methods, and when using embodiments of the present devices and systems, a pneumoperitoneum may be created in the cavity of interest to yield a relatively-open space within the cavity.

As shown in FIGS. 1 and 2, system 10 comprises an apparatus 34 and a medical device 38; the apparatus is configured to magnetically position the device with a body cavity of a patient. In some embodiments, apparatus 34 can be described as an exterior apparatus and device 38 as an interior device due the locations of their intended uses relative to patients. As shown, apparatus 34 can be positioned outside the cavity 18 near, adjacent to, and/or in contact with the exterior surface 30 of the patent 14. Device 38 is positionable (can be positioned), and is shown positioned, within the cavity 18 of the patient 14 and near, adjacent to, and/or in contact with the interior surface 26 of wall 22. Device 38 can be inserted or introduced into the cavity 18 in any suitable fashion. For example, the device 18 can be inserted into the cavity through a puncture (not shown) in wall 22, through a tube or trocar (not shown) extending into the cavity 18 through a puncture or natural orifice (not shown), or may be inserted into another portion of the patient 14 and moved into the cavity 18 with apparatus 34, such as by the methods described in this disclosure. If the cavity 18 is pressurized, device 38 can be inserted or introduced into the cavity 18 before or after the cavity 18 is pressurized.

Additionally, some embodiments of system 10 include a version of device 38 that has a tether 42 coupled to and extending away from the device 38. In the depicted embodiment, tether 42 extends from device 38 and out of the cavity 18, for example, through the opening (not shown) through which device 38 is introduced into the cavity 18. The tether 42 can be flexible and/or elongated. In some embodiments, the tether 42 can include one or more conduits for fluids that can be used, for example, for actuating a hydraulic cylinder or irrigating a region within the cavity 18. In some embodiments, the tether 42 can include one or more conductors for enabling electrical communication with the device 38. In some embodiments, the tether 42 can include one or more conduits for fluid and one or more conductors. In some embodiments, the tether does not include a conduit or conductor and, instead, includes a cord for positioning, moving, or removing device 38 from the cavity 18. The tether 14, for example, can be used to assist in positioning the device 34 while the device 34 is magnetically coupled to the apparatus 38, or to remove the device 34 from the cavity 18 when device 38 is not magnetically coupled to apparatus 34.

As is discussed in more detail below, apparatus 34 and device 38 can be configured to be magnetically couplable to one another such that device 38 can be positioned or moved within the cavity 18 by positioning or moving apparatus 34 outside the cavity 18. “Magnetically couplable” means capable of magnetically interacting so as to achieve a physical result without a direct physical connection. Examples of physical results are causing device 38 to move within the cavity 18 by moving apparatus 34 outside the cavity 18, and causing device 38 to remain in a position within the cavity 18 or in contact with the interior surface 26 of wall 22 by holding apparatus 34 in a corresponding position outside the cavity 18 or in contact with the exterior surface 30 of wall 22. Magnetic coupling can be achieved by configuring apparatus 34 and device 38 to cause a sufficient magnetic attractive force between them. For example, apparatus 34 can comprise one or more magnets (e.g., permanent magnets, electromagnets, or the like) and device 38 can comprise a ferromagnetic material. In some embodiments, apparatus 34 can comprise one or more magnets, and device 38 can comprise a ferromagnetic material, such that apparatus 34 attracts device 38 and device 38 is attracted to apparatus 34. In other embodiments, both apparatus 34 and device 38 can comprise one or more magnets such that apparatus 34 and device 38 attract each other.

The configuration of apparatus 34 and device 38 to cause a sufficient magnetic attractive force between them can be a configuration that results in a magnetic attractive force that is large or strong enough to compensate for a variety of other factors (such as the thickness of any tissue between them) or forces that may impede a desired physical result or desired function. For example, when apparatus 34 and device 38 are magnetically coupled as shown, with each contacting a respective surface 26 or 30 of wall 22, the magnetic force between them can compress wall 22 to some degree such that wall 22 exerts a spring or expansive force against apparatus 34 and device 38, and such that any movement of apparatus 34 and device 38 requires an adjacent portion of wall 22 to be similarly compressed. Apparatus 34 and device 38 can be configured to overcome such an impeding force to the movement of device 38 with apparatus 34. Another force that the magnetic attractive force between the two may have to overcome is any friction that exists between either and the surface, if any, that it contacts during a procedure (such as apparatus 34 contacting a patient's skin) Another force that the magnetic attractive force between the two may have to overcome is the force associated with the weight and/or tension of the tether 42 and/or frictional forces on the tether 42 that may resist, impede, or affect movement or positioning of device 38 using apparatus 34.

In some embodiments, device 38 can be inserted into cavity 18 through an access port having a suitable internal diameter. Such access ports includes those created using a conventional laparoscopic trocar, gel ports, those created by incision (e.g., abdominal incision), and natural orifices. Device 38 can be pushed through the access port with any elongated instrument such as, for example, a surgical instrument such as a laparoscopic grasper or a flexible endoscope.

In embodiments where the tether 42 is connectable to a power source or a hydraulic source (not shown), the tether can be connected to the power source or the hydraulic source (which may also be described as a fluid source) either before or after it is connected to device 38.

In some embodiments, when device 38 is disposed within cavity 18, device 38 can be magnetically coupled to apparatus 34. This can serve several purposes including, for example, to permit a user to move device 38 within cavity 18 by moving apparatus 34 outside cavity 18. The magnetic coupling between the two can be affected by a number of factors, including the distance between them. For example, the magnetic attractive force between device 38 and apparatus 34 increases as the distance between them decreases. As a result, in some embodiments, the magnetic coupling can be facilitated by temporarily compressing the tissue (e.g., the abdominal wall) separating them. For example, after device 38 has been inserted into cavity 18, a user (such as a surgeon) can push down on apparatus 34 (and wall 22) and into cavity 18 until apparatus 34 and device 38 magnetically couple.

In FIGS. 1 and 2, apparatus 34 and device 38 are shown at a coupling distance from one another and magnetically coupled to one another such that device 38 can be moved within the cavity 18 by moving apparatus 34 outside the outside wall 22. The “coupling distance” between two structures (e.g., apparatus 34 and device 38) is defined as a distance between the closest portions of the structures at which the magnetic attractive force between them is great enough to permit them to function as desired for a given application.

The “maximum coupling distance” between two structures (e.g., apparatus 34 and device 38) is defined as the greatest distance between the closest portions of the structures at which the magnetic attractive force between them is great enough to permit them to function as desired for a given application. Factors such as the thickness and composition of the matter (e.g., human tissue) separating them can affect the coupling distance and the maximum coupling distance for a given application. For example, in the embodiment shown in FIGS. 1 and 2, the maximum coupling distance between apparatus 34 and device 38 is the maximum distance between them at which the magnetic attractive force is still strong enough to overcome the weight of device 38, the force caused by compression of wall 22, the frictional forces caused by contact with wall 22, and any other forces necessary to permit device 38 to be moved within cavity 18 by moving apparatus 34 outside wall 22. In some embodiments, apparatus 34 and device 38 can be configured to be magnetically couplable such that when within a certain coupling distance of one another the magnetic attractive force between them is strong enough to support the weight of device 38 in a fixed position and hold device 38 in contact with the interior surface 26 of wall 22, but not strong enough to permit device 38 to be moved within the cavity 18 by moving apparatus 34 outside wall 22.

In some embodiments, apparatus 34 and device 38 can be configured to have a minimum magnetic attractive force at a certain distance. For example, in some embodiments, apparatus 34 and device 38 can be configured such that at a distance of 50 millimeters between the closest portions of apparatus 34 and device 38, the magnetic attractive force between apparatus 34 and device 38 is at least about: 20 grams, 25 grams, 30 grams, 35 grams, 40 grams, or 45 grams. In some embodiments, apparatus 34 and device 38 can be configured such that at a distance of about 30 millimeters between the closest portions of apparatus 34 and device 38, the magnetic attractive force between them is at least about: 25 grams, 30 grams, 35 grams, 40 grams, 45 grams, 50 grams, 55 grams, 60 grams, 65 grams, 70 grams, 80 grams, 90 grams, 100 grams, 120 grams, 140 grams, 160 grams, 180 grams, or 200 grams. In some embodiments, apparatus 34 and device 38 can be configured such that at a distance of about 15 millimeters between the closest portions of apparatus 34 and device 38, the magnetic attractive force between them is at least about: 200 grams, 250 grams, 300 grams, 350 grams, 400 grams, 45 grams, 500 grams, 550 grams, 600 grams, 650 grams, 700 grams, 800 grams, 900 grams, or 1000 grams. In some embodiments, apparatus 34 and device 38 can be configured such that at a distance of about 10 millimeters between the closest portions of apparatus 34 and device 38, the magnetic attractive force between them is at least about: 2000 grams, 2200 grams, 2400 grams, 2600 grams, 2800 grams, 3000 grams, 3200 grams, 3400 grams, 3600 grams, 3800 grams, or 4000 grams. These distances may be coupling distances or maximum coupling distances for some embodiments.

In some embodiments, apparatus 34 includes two magnetic field sources, where one of the magnetic field sources is a coupling magnetic field source that is relatively larger than the other or has a relatively stronger magnetic field than the other and therefore generates the majority of the magnetic attractive force, and the other of the magnetic field sources is relatively smaller than the other or has a relatively weaker magnetic field than the other and therefore generates a minority of the magnetic attractive force.

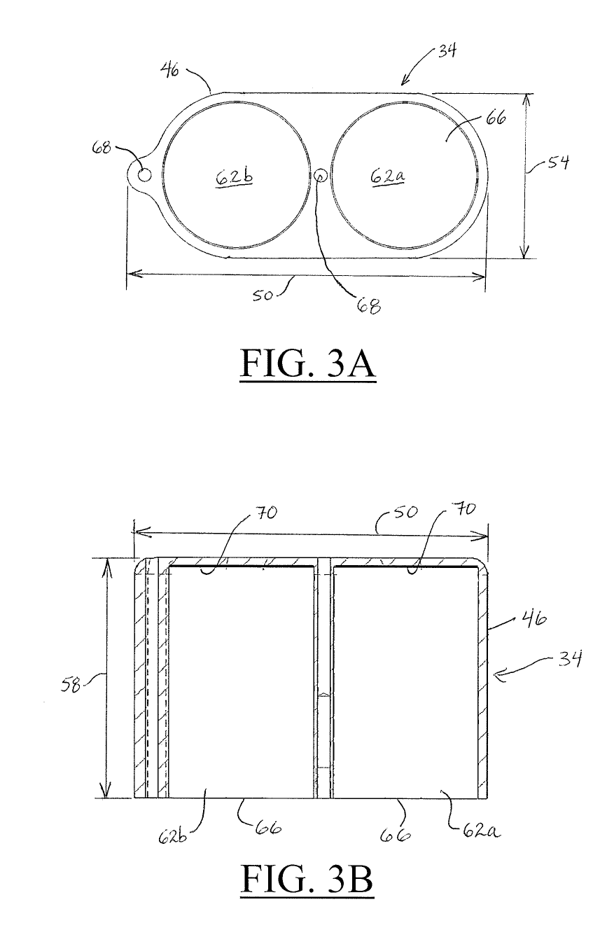

Referring now to FIGS. 3A and 3B, a bottom view and a side cross-sectional view are shown, respectively, of an embodiment of apparatus 34. Apparatus 34 has a width 50, a depth 54, and a height 58, and includes a housing 46. The apparatus (and, more specifically, housing 46) is configured to support, directly or indirectly, at least one magnetic assembly in the form of one or more magnetic field sources. In the embodiments shown, apparatus 34 is shown as including a first magnetic field source 62a and a second magnetic field source 62b. Each magnetic field source 62a, 62b has a coupling end 66 and a distal end 70. As described in more detail below, the coupling ends face device 38 when apparatus 34 and device 38 are magnetically coupled. The depicted embodiment of housing 46 of apparatus 34 also includes a pair of guide holes 68 extending through housing 46 for guiding, holding, or supporting various other devices or apparatuses, as described in more detail below. In other embodiments, the housing of apparatus 34 can have any other suitable number of guide holes 68 such as, for example, zero, one, three, four, five, or more guide holes 68. In some embodiments, housing 46 comprises a material that is minimally reactive to a magnetic field such as, for example, plastic, polymer, fiberglass, or the like. In other embodiments, housing 46 can be omitted or can be integral with the magnetic field sources such that the apparatus is, itself, a magnetic assembly comprising a magnetic field source.

Width 50, depth 54, and height 58 of a given embodiment of apparatus 34 can each be any size suited to the relevant application. In some embodiments, width 50 can be less than about 2.75 inches, depth 54 can be less than about 1.75 inches, and height 58 can be less than about 2.5 inches. Additionally, in some embodiments, width 50 can be less than about any of: 2 inches, 2.1 inches, 2.2 inches, 2.3 inches, 2.4 inches, 2.5 inches, 2.6 inches, 2.7 inches, 2.8 inches, 2.9 inches, or 3 inches; depth 54 can be less than about any of: 1 inch, 1.1 inches, 1.2 inches, 1.3 inches, 1.4 inches, 1.5 inches, 1.6 inches, 1.7 inches, 1.8 inches, 1.9 inches, or 2 inches; and height 58 can be less than about any of: 1.6 inches, 1.8 inches, 2 inches, 2.1 inches, 2.2 inches, 2.3 inches, 2.4 inches, 2.5 inches, 2.6 inches, 2.7 inches, 2.8 inches, 2.9 inches, 3 inches, 3.2 inches, 3.4 inches, 3.6 inches, 3.8 inches, or 4 inches.

In some embodiments, it can be useful to define a “coupling area” of apparatus 34. The “coupling area” for any given shape of apparatus 34 generally corresponds to the cross-sectional area of a portion of apparatus 34 proximal to the coupling ends of the magnetic field sources, and is no larger than necessary to circumscribe the same cross-sectional area with either a circle or rectangle. For example, in the embodiment shown, the coupling area can be defined as width 50 times depth 54. Thus, in one embodiment of apparatus 34 where width 50 is about 2.5 inches and depth 54 is about 1.5 inches, the coupling area is about 3.75 square inches. In other embodiments, the coupling area can be less than about any of: 3 square inches, 3.2 square inches, 3.4 square inches, 3.6 square inches, 3.8 square inches, 4 square inches, 4.2 square inches, 4.4 square inches, 4.6 square inches, 4.8 square inches, 5 square inches, 5.5 square inches, 6 square inches, 6.5 square inches, 7 square inches, 7.5 square inches, or 8 square inches.

In some embodiments, the volume of space occupied by apparatus 34 (which can be referred to as the volume of the apparatus) can be less than about any of: 64 cubic inches, 56 cubic inches, 48 cubic inches, 40 cubic inches, 32 cubic inches, 24 cubic inches, 16 cubic inches, 15 cubic inches, 14 cubic inches, 13 cubic inches, 12 cubic inches, 11 cubic inches, 10 cubic inches, 9 cubic inches, or 8 cubic inches.

Magnets, in general, have a north pole (the N pole) and a south pole (the S pole). In some embodiments, apparatus 34 can be configured (and, more specifically, its magnetic field sources can be configured) such that the coupling end 66 of each magnetic field source is the N pole and the distal end 70 of each magnetic field source is the S pole. In other embodiments, the magnetic field sources can be configured such that the coupling end 66 of each magnetic field source is the S pole and the distal end 70 of each magnetic field source is the N pole. In other embodiments, the magnetic field sources can be configured such that the coupling end of the first magnetic field source 62a is the N pole and the recessed end of the first magnetic field source 62a is the S pole, and the coupling end of the second magnetic field source 62b is the S pole and the recessed end of the second magnetic field source 62b is the N pole. In other embodiments, the magnetic field sources can be configured such that the coupling end of the first magnetic field source 62a is the S pole and its recessed end is the N pole, and the coupling end of the second magnetic field source 62b is the N pole and its recessed end is the S pole.

In the embodiment shown, each magnetic field source includes a solid cylindrical magnet having a circular cross section. In other embodiments, each magnetic field source can have any suitable cross-sectional shape such as, for example, rectangular, square, triangular, fanciful, or the like. In some embodiments, each magnetic field source comprises any of: any suitable number of magnets such as, for example, one, two, three, four, five, six, seven, eight, nine, ten, or more magnets; any suitable number of electromagnets such as, for example, one, two, three, four, five, six, seven, eight, nine, ten or more electromagnets; any suitable number of pieces of ferromagnetic material such as, for example, one, two, three, four, five, six, seven, eight, nine, ten or more pieces of ferromagnetic material; any suitable number of pieces of paramagnetic material such as, for example, one, two, three, four, five, six, seven, eight, nine, ten or more pieces of paramagnetic material; or any suitable combination of magnets, electromagnets, pieces of ferromagnetic material, and/or pieces of paramagnetic material.

In some embodiments, each magnetic field source can include four cylindrical magnets (not shown) positioned in end-to-end in linear relation to one another, with each magnet having a height of about 0.5 inch and a circular cross-section that has a diameter of about 1 inch. In these embodiments, the magnets can be arranged such that the N pole of each magnet faces the S pole of the next adjacent magnet such that the magnets are attracted to one another and not repulsed.

Examples of suitable magnets can include: flexible magnets; Ferrite, such as can comprise Barium or Strontium; AlNiCo, such as can comprise Aluminum, Nickel, and Cobalt; SmCo, such as can comprise Samarium and Cobalt and may be referred to as rare-earth magnets; and NdFeB, such as can comprise Neodymium, Iron, and Boron. In some embodiments, it can be desirable to use magnets of a specified grade, for example, grade 40, grade 50, or the like. Such suitable magnets are currently available from a number of suppliers, for example, Magnet Sales & Manufacturing Inc., 11248 Playa Court, Culver City, Calif. 90230 USA; Amazing Magnets, 3943 Irvine Blvd. #92, Irvine, Calif. 92602; and K & J Magnetics Inc., 2110 Ashton Dr. Suite 1A, Jamison, Pa. 18929. In some embodiments, one or more magnetic field sources can comprise ferrous materials (e.g., steel) and/or paramagnetic materials (e.g., aluminum, manganese, platinum).

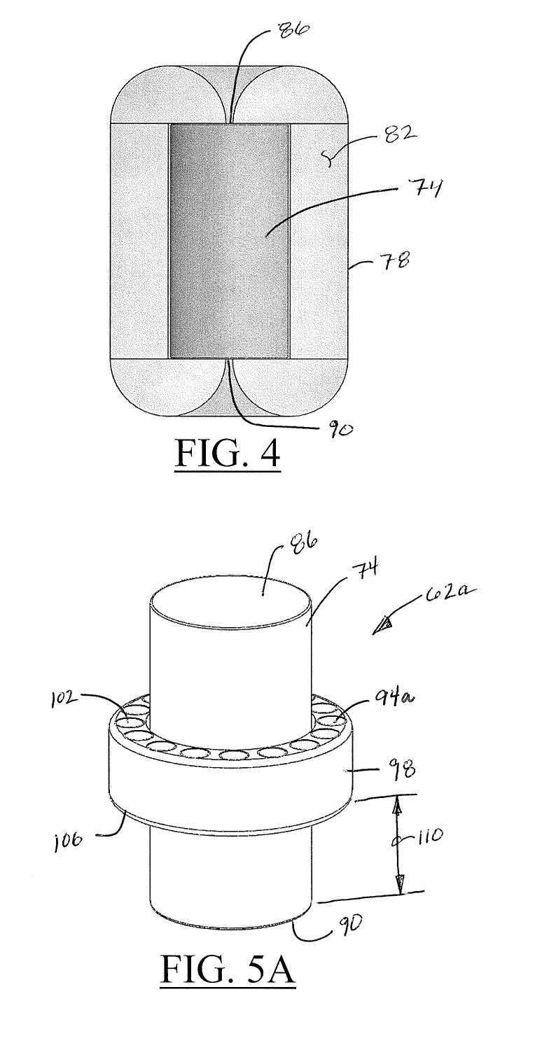

Referring now to FIG. 4, a side view is shown of a cylindrical magnet 74 that may be used as at least part of one of the present magnetic field sources. Field lines 78 conceptually illustrate the magnetic field 82 of magnet 74. Magnet 74 has a first end 86 and a second end 90. As described above, magnet 74 has a N pole and an S pole. For a cylindrical magnet having a circular cross-sectional shape, such as magnet 74, the N and S poles are generally aligned with the axis passing through the center of the circular cross-sectional shape. For example, when first end 86 is the N pole, second end 90 is generally the S pole; and where first end 86 is the S pole, second end 90 is generally the N pole. As conceptually illustrated by field lines 78, in the absence of external influences, magnetic field 82 is generally evenly distributed about magnet 74 and flows through the N and S poles. Although magnet 74 is shown as a single cylindrical cylinder, in some embodiments (not shown), magnet 74 can comprise a plurality of, for example, two, three, four, or more, shorter cylindrical magnets oriented in a linear configuration to form magnet 74. In such an embodiment, each shorter magnet will similarly have an N and a S pole, and can be oriented such that the S pole of each shorter magnet is adjacent to the N pole of the next adjacent shorter magnet, such that each S pole attracts and is attracted to the next adjacent N pole.

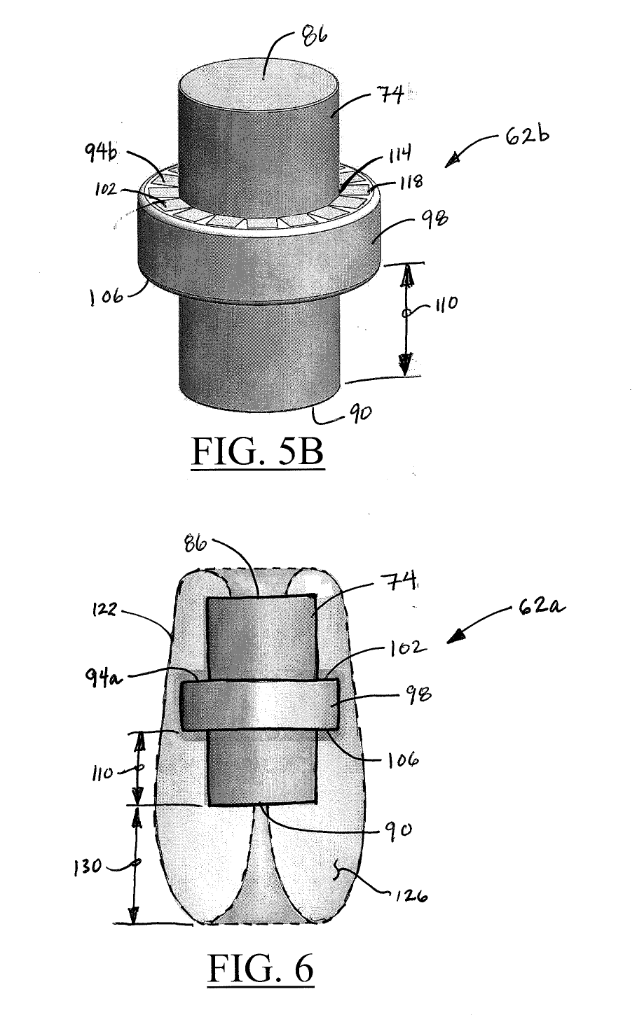

Referring now to FIGS. 5A and 5B, perspective views are shown of two embodiments, respectively, of magnetic field sources 62a and 62b that can be used with various embodiments of apparatus 34, such as the embodiment shown in FIGS. 1-3B. As shown in FIG. 5A, magnetic field source 62a is an embodiment of a magnetic assembly that includes a primary magnetic field source in the form of primary magnet 74, and a plurality of peripheral magnetic field sources in the form of peripheral magnets 94a disposed about primary magnet 74 in support ring 98. Primary magnet 74 can be configured similarly to (including identically as) the embodiment of magnet 74 of FIG. 4. In other embodiments, primary magnet 74 can be configured in any suitable fashion described in this disclosure or otherwise known in the art. As shown, each peripheral magnet 94a can be cylindrical and smaller than primary magnet 74. In other embodiments, the peripheral magnets can each have any suitable shape or size that permits magnetic field source 62a to function as described in more detail below. Each peripheral magnet 94a can have a first end 102 oriented toward (e.g., facing substantially the same direction as) first end 86 of primary magnet 74, and a second end 106 oriented toward second end 90 of primary magnet 74. Support ring 98 can be configured to support or hold peripheral magnets 94a in fixed relation to one another. In some embodiments, support ring 98 can also be configured to support or hold peripheral magnets 94a in fixed relation to primary magnet 74, such as, for example, a distance 110 from the second end 90 of the primary magnet 62a. In other embodiments, the support ring 98 can be configured to support or hold the peripheral magnets 94a in slidable relation to primary magnet 74 such that, for example, distance 110 is adjustable by sliding support ring 98 relative to primary magnet 74.

In some embodiments, support ring 98 can be configured to be slidable by hand to adjust distance 110. In other embodiments, the support ring can be configured to be threaded onto or about the primary magnet such that distance 110 is adjustable by rotating the support ring relative to the primary magnet. In some embodiments, distance 110 can be adjusted to a value that is predetermined as a function of a patient's body mass index (BMI), as a function of the thickness of the wall through which apparatus 34 and device 38 are to be magnetically coupled, or as a function of any other useful parameter. In some embodiments, one or both of apparatus 34 and device 38 can be provided with one or more sensors (e.g., strain gauges) to measure the attractive force between the apparatus and the device and/or send a signal indicating that the distance 110 should be adjusted to increase or decrease the attractive force (e.g., to achieve one or more of the force-distance combinations discussed above). In some embodiment, the signal can be sent to a display for indicating to a user (such as a doctor) in a form perceivable by the user (e.g., light, screen, audible alarm, or the like) that the distance 110 should be adjusted to increase or decrease the attractive force. In other embodiments, the signal can be sent to a processor or the like, to trigger an automated adjustment of distance 110 to increase or decrease the attractive force. In some embodiments, magnetic field source 62a can be configured with or as apparatus 34 such that second end 90 of primary magnet 74 is coupling end 66 of magnetic field source 62a.

As described above for magnets generally, each peripheral magnet 94a can have an N pole and an S pole. In some embodiments, the N pole of a given peripheral magnet 94a can be aligned with its first end 102 and the S pole can be aligned with its second end 106, or vice-versa. In some embodiments, the N pole of a given peripheral magnet 94a can be oriented radially inward toward primary magnet 74. In some embodiments, all of the peripheral magnets 94a can be similarly aligned such that the N pole of each is aligned with the first end 102 of each, or such that the S pole of each is aligned with the first end 102 of each.

As shown in FIG. 5B, peripheral magnets 94b can be rectangular in shape, and each rectangular peripheral magnet 94b can have a first end 102, a second end 106, an interior surface 114, and an exterior surface 118. As shown, the interior surface of each peripheral magnet can be oriented toward primary magnet 74 and the exterior surface of each peripheral magnet can be oriented away from primary magnet 74. The N and S poles of each rectangular peripheral magnet 94b can be oriented in any suitable way. For example, in some embodiments, the N pole of each is oriented toward first end 102 and the S pole of each is oriented toward second end 106. In other embodiments, the N pole of each is oriented toward interior surface 114 and the S pole of each is oriented toward exterior surface 118.

Referring now to FIG. 6, a side view is shown of the embodiment of magnetic field source 62a shown in FIG. 5A with field lines 122 conceptually illustrating the magnetic field 126 of the magnetic field source. In the embodiment shown, primary magnet 74 has its N pole aligned with first end 86 and its S pole aligned with second end 90; and each peripheral magnet 94a has its N pole aligned with its first end 102 and its S pole aligned with its second end 106, such that primary magnet 74 is in an N-S configuration and peripheral magnets 94a are in a similar N-S configuration, thus yielding a primary/peripheral configuration of N-S/N-S. In this configuration, the S pole of each peripheral magnet 94a repels the S pole of primary magnet 74, and the N pole of each peripheral magnet 94a repels the N pole of primary magnet 74, such that at least a portion of the magnetic field 126 is effectively compressed radially inward along at least a portion of the length of primary magnet 74 so as to force magnetic field 126 away from second end 90 of primary magnet 74. For example, this projected or “focused” magnetic field 126 can project a distance 130 beyond the second end 90 that is greater than the distance the magnetic field 126 would extend in the absence of the peripheral magnets 94a. In the embodiment shown, the second end 90 of primary magnet 74 can be the coupling end 66 of magnetic field source 62a such that projected magnet field 126 increases (relative to not using peripheral magnets 94a) the maximum coupling distance that can be achieved between apparatus 34 and device 38.

Additionally, when, as in some embodiments, the support ring is slidable or otherwise movable relative to the primary magnet the distance 130 can be adjusted by moving the support ring relative to the primary magnet so as to adjust the distance 110. Although this focusing effect is described with reference to magnetic field source 62a shown in FIG. 5A, support ring 98 that is part of magnetic field source 62b shown in FIG. 5B can also be configured to be slidable relative to the primary magnet shown in FIG. 5B to achieve the same type of projected magnetic field. In other embodiments of the magnetic field sources shown in FIGS. 5A and 5B can have a primary/peripheral configuration of N-S/S-N such that the N pole of the peripheral magnets 94a attracts the S pole of primary magnet 74 to cause the magnetic field of primary magnet 74 to follow the path of least resistance through the peripheral magnets 94a.

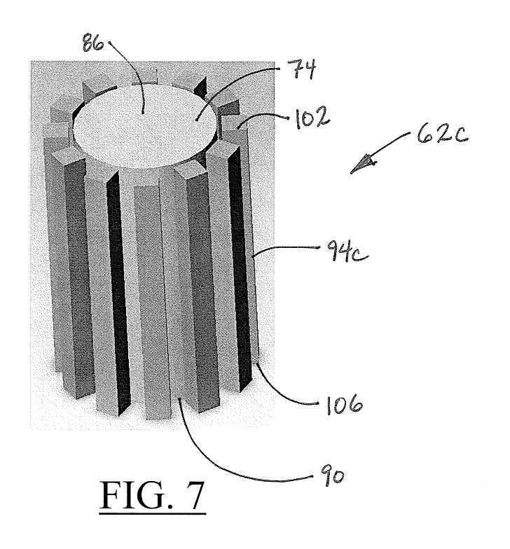

Referring now to FIG. 7, a perspective view is shown of a third embodiment of one of the present magnetic assemblies in the form of magnetic field source 62c, which can be used with or as apparatus 34 shown in FIGS. 1-3B. Magnetic field source 62c comprises primary magnet 74 and a plurality of peripheral magnets 94c disposed about primary magnet 74. In contrast to the embodiments described above, the peripheral magnets 94c are about the same length as primary magnet 74, such that the first end 102 of each peripheral magnet is adjacent to (or substantially coplanar with) the first end 86 of primary magnet 74, and the second end 106 of each peripheral magnet is adjacent to (or substantially coplanar with) the second end 90 of primary magnet 74. The peripheral magnets 94c can be supported or held in fixed relation to one another, and/or in fixed or sliding relation to primary magnet 74, by any suitable means such as, for example, an adhesive, a housing (not shown), or one or more support rings (e.g., support ring 98 shown in FIGS. 5A and 5B).

In one embodiment of magnetic field source 62c, the N pole of primary magnet 74 is aligned with first end 86 and the S pole is aligned with second end 90; and each peripheral magnet 94c has its N pole aligned with its first end 102 and its S pole aligned with its second end 106, such that primary magnet 74 is in an N-S configuration and peripheral magnets 94c are in a similar N-S configuration, thus yielding a primary/peripheral configuration of N-S/N-S. In this configuration, the S pole of each of the peripheral magnets 94a repels the S pole of primary magnet 74, and the N pole of each of the peripheral magnets 94c repels the N pole of primary magnet 74, such that at least a portion of magnetic field 126 is effectively compressed radially inward along at least a portion of the length of primary magnet 74 so as to force the magnetic field away from second end 90 of primary magnet 74, as described above with reference to FIG. 6. In other embodiments, the magnetic field source 62c can have a primary/peripheral configuration of N-S/S-N such that the N pole of each of the peripheral magnets attracts the S pole of primary magnet 74 to cause the magnetic field of primary magnet 74 to follow a path of least resistance through the peripheral magnets 94c.

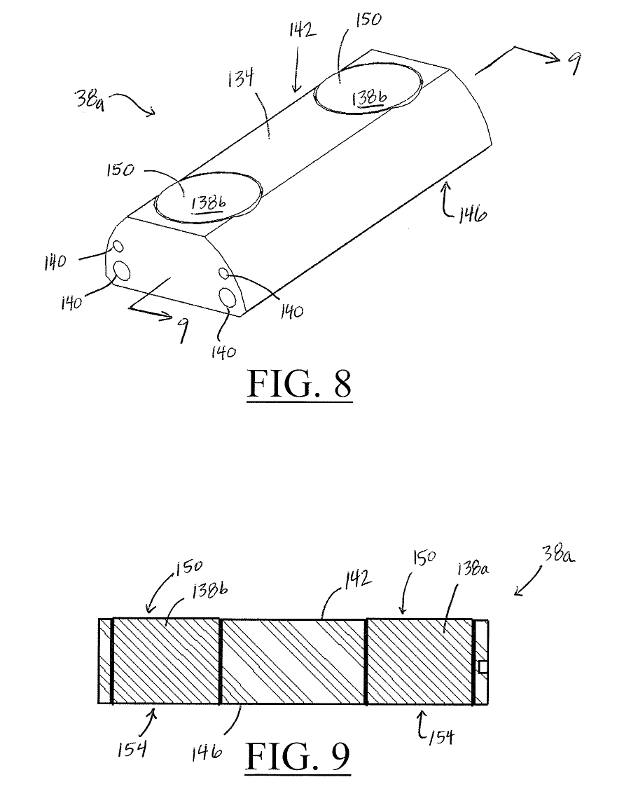

Referring now to FIGS. 8 and 9, FIG. 8 depicts device 38a, another embodiment of one of the present medical devices that can be moved within a body cavity using one of the present apparatuses to which it is magnetically coupled, and which can also be used as part of one of the present systems. In embodiments of the present medical devices and systems in which the medical device (e.g., devices 38a, 38b, 38c, 38d, 38d-1, 38e, 38f, 38g, 38h, 38i, 38j, 38k) includes an arm, a tool, a light emitting diode (LED), or the like that is coupled to the structure shown, for example, in FIGS. 8 and 9, that structure may be referred to as a “platform.”

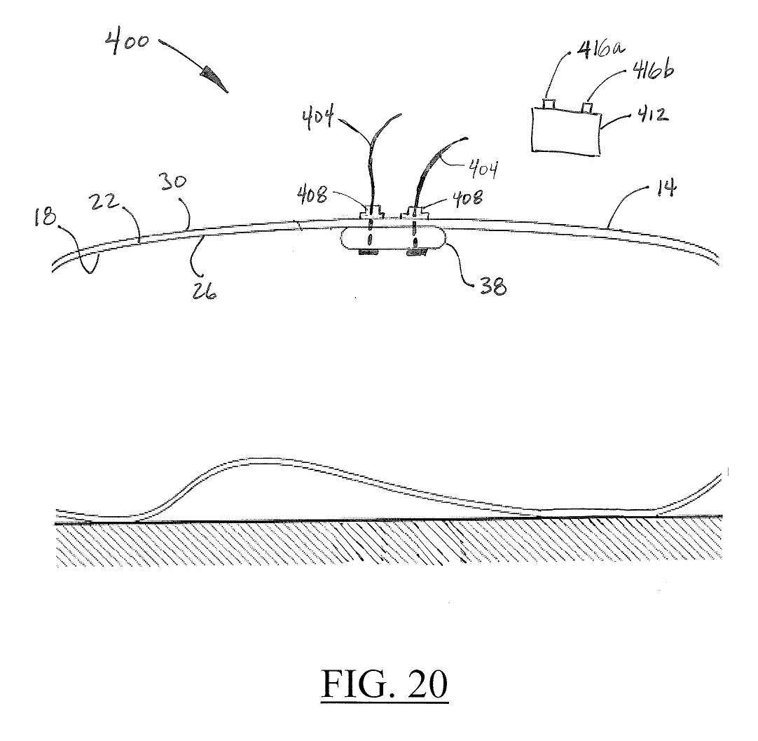

FIG. 9 depicts a cross-sectional view of device 38a taken along line 9-9 in FIG. 8. In the embodiment shown, device 38a comprises a magnetically attractive material. More specifically, device 38a includes housing 134 and two magnetically-attractive members (in this case, first member 138a and second member 138b), which are supported by (e.g., coupled to) housing 134. In the embodiment shown, device 38a has holes 140 extending into at least a portion of device 38a and configured, for example, to enable coupling of a tool (not shown), a tether 42, or the like by way of a fastener, adhesive, or the like. One or more of holes 140 can be configured to hold all or a portion of an insert or attachment, such as enables device 38a to function, for example, in system 400 or with apparatus 404 (described below with reference to FIGS. 20-29). For example, holes 140 can be used to anchor a cone-shaped nose (not shown) to facilitate insertion of the device 38a into a body cavity, In other embodiments of the present devices, holes 140 may be omitted altogether or configured in different or additional ways. Device 38a has a coupling side 142 and a working side 146. Device 38a can be part of embodiments of the present systems that include an embodiment of apparatus 34. Device 38a can be configured such that coupling side 142 faces an embodiment of apparatus 34, and such that working side 146 faces away from apparatus 34, when apparatus 34 and device 38a are magnetically coupled to each other. Housing 134 can support or hold members 138a and 138b in fixed relation to one another. Each magnetically-attractive member has a coupling end 150 oriented toward coupling side 142 of device 38a and a distal end 154 oriented toward working side 146 of device 38a.

Members 138a and 138b can comprise any suitable material that is magnetically attracted to the magnetic field sources 62a, 62b of apparatus 34. Examples of such material include, for example, a magnet, a ferromagnetic material, and a paramagnetic material. In some embodiments, one or both of apparatus 34 and device 38a are configured such that that the magnetic field sources of the apparatus can each be aligned with a different magnetically-attractive member of device 38a, meaning that an axis can be substantially centered in and run lengthwise through a given aligned pair comprising a magnetic field source of the apparatus and a magnetically-attractive member of the device. In some embodiments of the present devices, e.g., device 38a, each member 138a, 138b comprises a cylindrical magnet having a height of about 0.25 inches, and a circular cross-section with a diameter of about 0.375 inches. In other embodiments, each member comprise a cylindrical magnet having a height of about any of 0.15 inches, 0.16 inches, 0.17 inches, 0.18 inches, 0.19 inches, 0.20 inches, or 0.21 inches; and a circular cross-section with a diameter of about any of: 0.25 inches, 0.3 inches, 0.35 inches, 0.375 inches, 0.4 inches, 0.45 inches, 0.5 inches, 0.55 inches, 0.6 inches, 0.625 inches, or 0.65 inches. In some embodiments, each member comprises a plurality of magnets of varying sizes or shapes, for example, five cylindrical magnets having a circular cross-section, two with a height of about 0.6 inches and a diameter of about 0.375 inches, and three with a height of about 0.6 inches and a diameter of about 0.5 inches; four cylindrical magnets having a circular cross section, one with a height of about 0.06 inches and a diameter of about 0.5 inches, and three with a height of about 0.6 inches and a diameter of about 0.625 inches. In other embodiments, members 138a, 138b include any suitable cross-sectional shape, dimension, or number of magnets, or volumes of ferromagnetic or paramagnetic materials.

In embodiments of the present devices, e.g. device 38a, where members 138a, 138b include magnets, each member will generally have an N pole and an S pole. In some of these embodiments, first member 138a has its N pole oriented toward coupling end 150 and its S pole oriented toward distal end 154, and second member 138b has its S pole oriented toward its coupling end 150 and its N pole oriented toward its distal end 154, such that the members 138a, 138b are in an N-S/S-N configuration. In others of these embodiments, first member 138a has its S pole oriented toward coupling end 150 and its N pole oriented toward distal end 154, and second member 138b has its N pole oriented toward its coupling end 150 and its S pole oriented toward its distal end 154, such that the members 138a, 138b are in an S-N/N-S configuration.

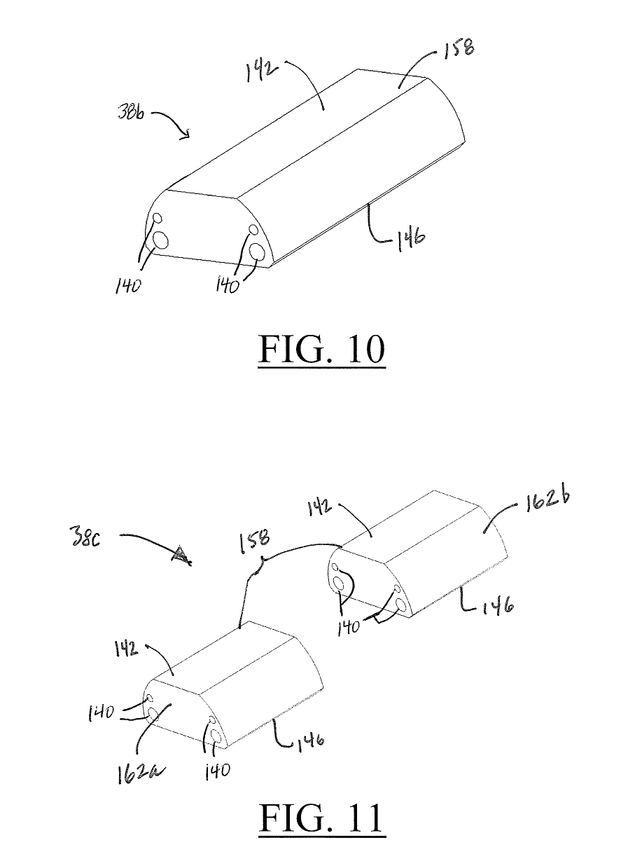

Referring now to FIG. 10, a perspective view is shown of medical device 38b, another embodiment of one of the present medical devices, which can also be used as part of one of the present systems. Device 38b includes a body 158 that is a magnet. The magnet can be manufactured to maximize magnet volume and magnetic coupling force. This embodiment of device 38b may be characterized as a “whole-body” magnet configuration. In some such versions of device 38b, body 158 can be configured such that its coupling side 142 is its N pole and its working side 146 is its S pole. In other versions, device 38b is configured such that coupling side 142 of body 158 is the S pole and working side 146 is the N pole.

Referring now to FIG. 11, a perspective view is shown of medical device 38c, another embodiment of one of the present medical devices, which can also be used as part of one of the present systems. The body 158 of device 38c has two body portions 162a and 162b, each being a magnet. Although the two body portions 162a and 162b are shown spaced apart, they can be coupled or linked together, for example, by way of holes 140, prior to and during use such that they are supported or held in fixed relation to one another by, for example, screws, bolts, rivets, adhesive, rods, tabs, or by a magnetic attractive force arising between them. In some versions of device 38c, body 150 can be configured such that the N pole of one of the body portions 162a, 162b is oriented toward its coupling side 142 and the S pole is oriented toward its working side 146, and such that the S pole of the other of the body portions 162a, 162b is oriented toward its coupling side 142 and the N pole is oriented toward its working side 146. In other embodiments, the N poles of both body portions 162a, 162b are oriented toward coupling sides 142 and the S poles are oriented toward working sides 146. In other embodiments, the S poles of both body portions are oriented toward coupling sides 142 and the N poles are oriented toward working sides 146.

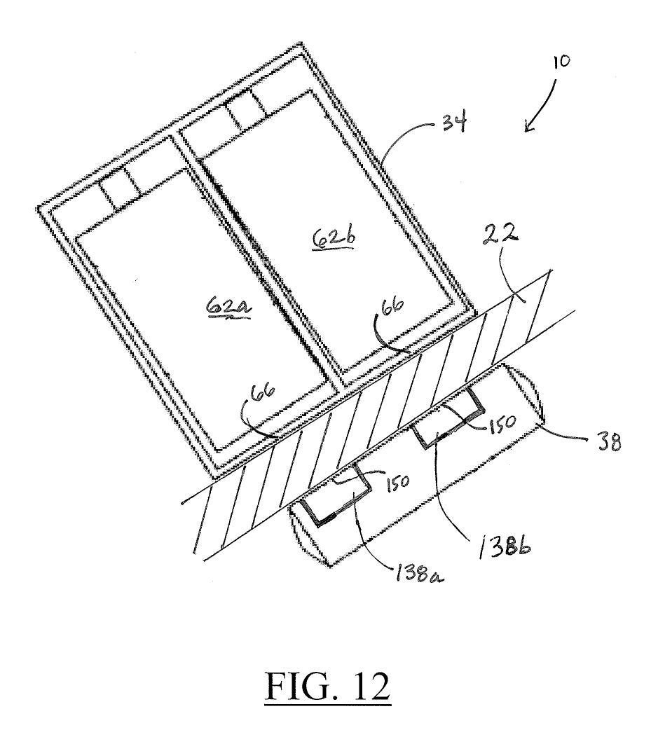

Referring now to FIG. 12, a pictorial side view is depicted of an embodiment of system 10 in which apparatus 34 and device 38 are magnetically coupled across a wall 22 of a patient with wall 22 shown in cross-section for clarity. As described above, the magnetic field sources 62a, 62b of apparatus 34 and the magnetically-attractive members 138a, 138b of device 38 can be configured in various ways. In one “consistent” configuration, the coupling ends 66 of both magnetic field sources 62a, 62b are configured to have the same polarity (e.g., both N poles or both S poles), such that the coupling ends 66 of the magnetic field sources 62a, 62b have an N-N configuration or orientation or an S-S configuration or orientation. In this “consistent” configuration, device 38 can be configured such that members 138a, 138b are magnets and coupling ends 150 of members 138a, 138b are oppositely oriented relative to coupling ends 66 of magnetic field sources 62. For example, where coupling ends 66 of the field sources 62a, 62b have an N-N configuration, members 138a, 138b of the device can have an S-S configuration, and where coupling ends 66 have an S-S configuration, coupling ends 150 can have an N-N configuration. In this way, magnetic field sources 62a, 62b and members 138a, 138b will be attracted to, and attract, each other such that the magnetic attractive forced can be maximized between apparatus 34 and device 38.

In another “alternating” configuration, coupling ends 66 of magnetic field sources 62a, 62b can be configured to have different polarities. For example, the N pole of first magnetic field source 62a can be oriented at coupling end 66 while the S pole of second magnetic field source 62b can be oriented at its coupling end 66, or vice versa, such that the coupling ends of the magnetic field sources have an N-S or S-N configuration. In this “alternating” configuration, device 38 can be configured such that members 138a, 138b are magnets that also have an alternating orientation. For example, coupling ends 150 of members 138a, 138b can have an N-S orientation or an S-N orientation. In this way, the coupling end 66 with an N pole primarily attracts and is attracted to the coupling end 150 having an S pole, and the coupling end 66 with an S pole primarily attracts and is attracted to the coupling end 150 having an N pole. Stated otherwise, each coupling end 66 attracts and is attracted to the coupling end 150 having an opposite polarity, and each coupling end 66 repels and is repelled by the coupling end 150 having a like polarity. As such, when in the “alternating” configuration, apparatus 34 and device 38 are attracted to one another in a specific relationship, such that when apparatus 34 and device 38 are magnetically coupled, control over or “tracking” of device 38 can be improved.

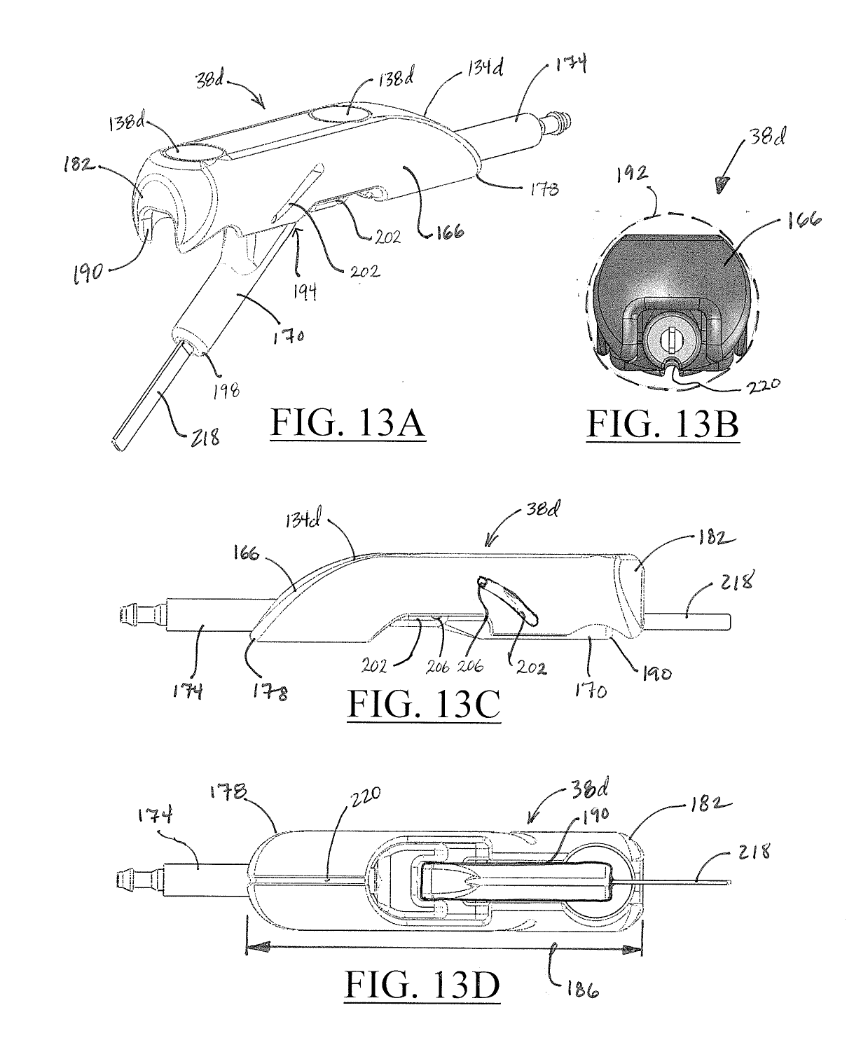

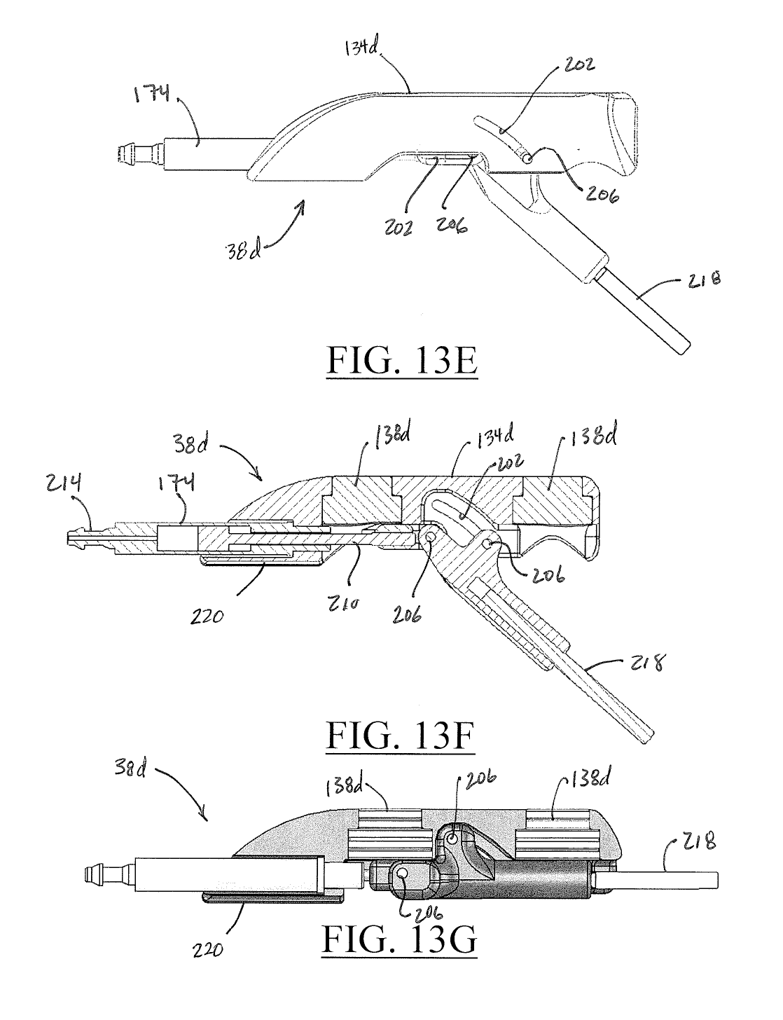

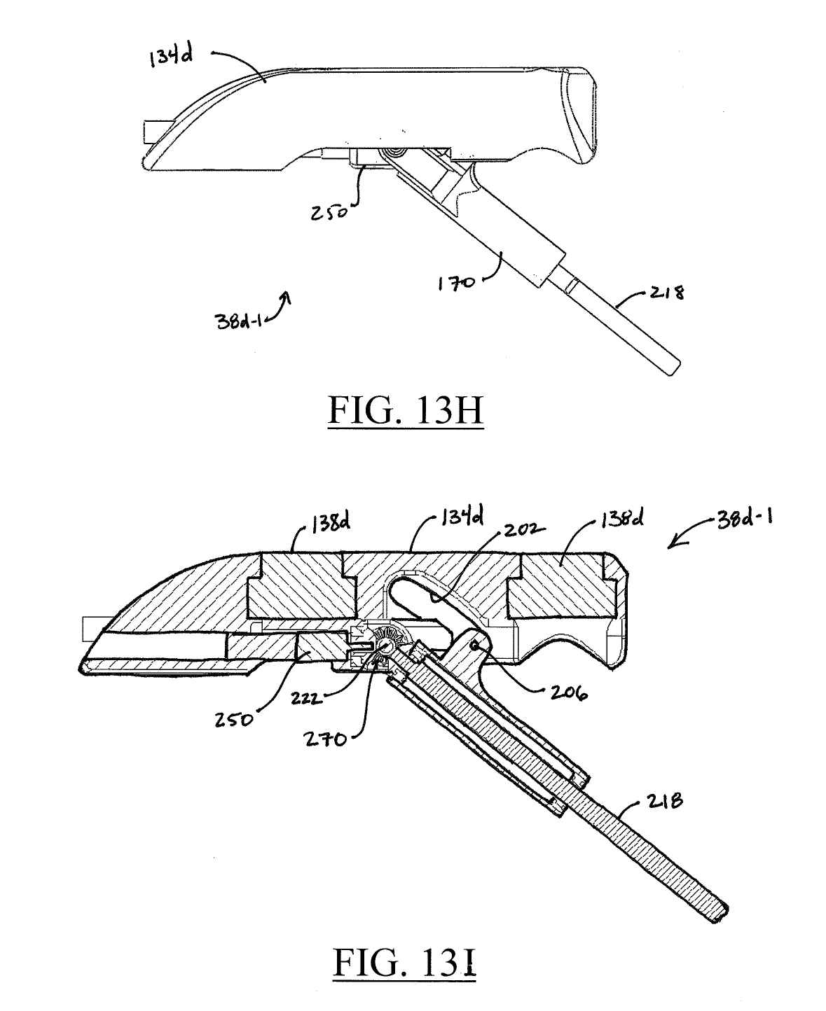

Referring now to FIGS. 13A-13G, various views are shown of medical device 38d, another embodiment of one of the present medical devices, which can also be used as part of one of the present systems. In the depicted embodiment, device 38d comprises a platform 166, an arm 170 that is coupled to the platform, and a cylinder 174 that is coupled to both the arm and the platform and that can be used to move the arm (as described in more detail below) from a collapsed position to an expanded position. As shown, platform 166 can comprise a housing 134d and can support one or more magnetically-attractive members 138d, as described above. Platform 166 has a proximal end 178, a distal end 182, and a length 186 extending between proximal end 178 and distal end 182. Platform 166 also has, in the depicted embodiment, a longitudinal recess 190 defined along at least a portion of length 186 of the platform. As shown in FIG. 13B, platform 166 can also have a maximum transverse perimeter 192. The “maximum transverse perimeter” of one of the present platforms is defined by the smallest circle or rectangle that can circumscribe the largest cross-section of the platform.

Arm 170 can have a proximal end 194 and a distal end 198. As shown, device 38d can be configured such proximal end 194 of arm 170 is distal to proximal end 178 of platform 166. The distance separating proximal ends 194 and 178 can be expressed as a percentage of the length of the platform from the platform's proximal end to its distal end, such as, for example, 1, 5, 10, 20, 30, 40, or 50 percent of the length of the platform, or any range or integer between 0 and 50 percent of the length of the platform. Arm 170 can also be coupled to platform 166 such that arm 170 is movable between (1) a collapsed position where distal end 198 of arm 170 is adjacent to platform 166, or where arm 170 is substantially parallel to platform 166, as shown in FIG. 13C, and (2) an expanded position where distal end 190 of arm 170 is spaced apart from platform 166, or where arm 170 is oriented at a non-zero angle to platform 166. As shown in this embodiment, arm 170 can be coupled to platform 166 by way of cam slots and pins. For example, in the embodiment shown, platform 166 includes a first cam slot 202 parallel to the longitudinal axis of platform 166 and extending transversely through the platform, and one or more additional cam slots 202 (two, in the depicted embodiment) that are spaced apart from and angularly disposed relative to the first cam slot 202 and that extend transversely through at least a portion of platform 166. In the embodiment shown, proximal end 194 of arm 170 can be coupled to the platform 166 by pins 206 extending into cam slots 202 and 202, such that in moving from the collapsed position of FIG. 13C to the expanded position of FIG. 13A, arm 170 moves both longitudinally in the direction of distal end 182 of platform 166 and angularly outward from platform 166. When arm 170 is in the collapsed position, the longitudinal axis of arm 170 is preferably disposed within the maximum transverse perimeter. Similarly, when arm 170 is in the collapsed position, at least a portion of the arm can be disposed within recess 190 such that a majority of the lateral sides of arm 170 is bordered by the platform such that platform 166 affords some protection to arm 170 during, for example, insertion into and removal from cavity 18.

As best shown in FIG. 13F, cylinder 174 can include a piston 210 and an inlet 214. Piston 210 can be coupled, directly or indirectly, to proximal end 194 of arm 170. Inlet 214 can be coupled to a fluid conduit (not shown) that runs through, with, or along tether 42 such that fluid can be delivered and removed, or pressurized and de-pressurized, to extend piston 210 toward distal end 182 of platform 166 such that arm 170 moves to the expanded position, and to retract piston 210 back toward proximal end 178 of platform 166 such that the arm moves to the collapsed position.

Device 38d can also include a tool 218, for example, a blade, a hook, a cautery tool, or any other tool that may be useful or advantageous for a medical procedure. In the embodiment shown, tool 218 is a cautery tool. Cautery tool 218 can be coupled to arm 170, for example, at or near the distal end 198 of the arm. Cautery tool 218 can be powered by way of a conductor (not shown) that runs through, with, or along the tether 42. Furthermore, during use of device 38d, the conductor can be positioned in notch or channel 220 located in the proximal portion of body 166 and visible, for example, in FIGS. 13B, 13D, 13F, and 13G. In some embodiments, cautery tool 218 can be positively charged with a high electric voltage, such as, for example, a voltage that is compatible with known electrosurgical units (e.g., up to 9,000 Volts peak-to-peak and/or 390 kHz sinusoidal), such that when cautery tool 218 contacts a grounded patient's flesh or tissue, the circuit completes and cautery tool 218 is able to cut or cauterize the flesh or tissue with relatively little force. Device 38d, as well as other embodiments of the present medical devices that include a cautery tool, can be configured such that when arm 170 is in the collapsed position, the distal end of cautery tool 218 is spaced apart (along a line parallel to the axes of both the tool and the platform) from the distal 182 end of the platform 166, such as by a distance of greater than or about any of: 0.1 inches, 0.2 inches, 0.3 inches, 0.4 inches, 0.5 inches, 0.6 inches, 0.7 inches, 0.8 inches, 0.9 inches, 1.0 inches, 1.2 inches, 1.4 inches, 1.6 inches, 1.8 inches, or 2 inches. Stated another way, device 38d can be configured such that when arm 170 is in the collapsed position, the distal end of tool 218 is located in a position that is spaced apart from the proximal end of the platform by a distance that is greater than the length of the platform. In some embodiments, tool 218 can be covered with a removable atraumatic tip or other cover (not shown) to, for example, facilitate insertion or removal of device 38d into or from cavity 18. When arm 170 is in the collapsed position, the longitudinal axis of cautery tool 218, or another tool 218, can be parallel to the longitudinal axis of arm 170 and can also be within the maximum transverse perimeter of platform 166.

In some embodiments, device 38d can be inserted into cavity 18 and magnetically coupled to apparatus 34, as described above. Once device 38d and apparatus 34 are magnetically coupled to each other, or device 38d is otherwise secured in position within cavity 18, a user can deploy or expand the tool (e.g., cautery tool 218) from the collapsed position (e.g. FIG. 13B, 13C) to an expanded position (e.g. FIG. 13A, 13D) by actuating cylinder 174 to extend piston 210 relative to cylinder 174. For example, cylinder 174 can be actuated by way of a hydraulic source (not shown), such as a syringe, a hand pump, a gas bottle (with a valve, a pump, or the like to control fluid flow), pressure regulator, or any other suitable source.

In some embodiments, when arm 170 is in an expanded position, the user can move device 38d to adjust its position within cavity 18 by moving magnetically coupled apparatus 34 outside cavity 18. In some embodiments, the user may further be able to move or adjust the pitch and yaw of device 38d by, for example, moving or adjusting the pitch and yaw of apparatus 34 where wall 22 is compliant enough to permit such pitch and yaw motion or adjustment. Embodiments of the present devices and systems can be configured such that when device 38d is in an operational position (e.g., cautery tool 218 is in a position that is acceptable to the user for performing a task within cavity 18), cautery tool 218 can be activated or electrified in any suitable manner, including, for example, through an electrosurgery unit (with or without a foot pedal), a power source, or the like. Embodiments of the present devices and systems can be configured such that cautery tool 218 can be powered and actuated by conventional methods and systems such as, for example, with a conventional cautery power supply. Such a power supply can be electrically-coupled to or in electrical communication with the cautery tool 218 in any suitable manner, including, for example, by way of a physical tether (e.g., tether 42 or apparatus 404, as described in more detail below). Embodiments of the present devices and systems can be configured such that a user can activate cautery tool 218 using a foot pedal, a switch, a voice-actuated activator, or any other suitable method, system, or device. Other embodiments of the present devices and systems can be configured such that cautery tool 218 can be deployed (e.g., arm 170 can be deployed from a collapsed to an expanded position) and/or controlled by way of a joystick or other relatively more-complicated user interface.

Referring now to FIGS. 13H and 13I, side and a cross-sectional views, respectively, are shown of medical device 38d-1, another embodiment of one of the present medical devices, which can also be used as part of one of the present systems. Medical device 38d-1 is similar in several respects to medical device 38d of FIGS. 13A-13G, so generally only the differences between them will be described here. In particular, rather than a cylinder (e.g., 174) device 38d-1 comprises a rotary motor 250 coupled in fixed relation to housing 134d. Further, arm 170 is rotatably coupled to housing 134d by a pin or axle 222, and arm 170 is coupled to motor 250 by bevel (or miter) gears 270, such that rotation of motor 250 can be configured to rotation of arm 170 around pin 222 (e.g., to move arm 170 between collapsed and deployed positions).

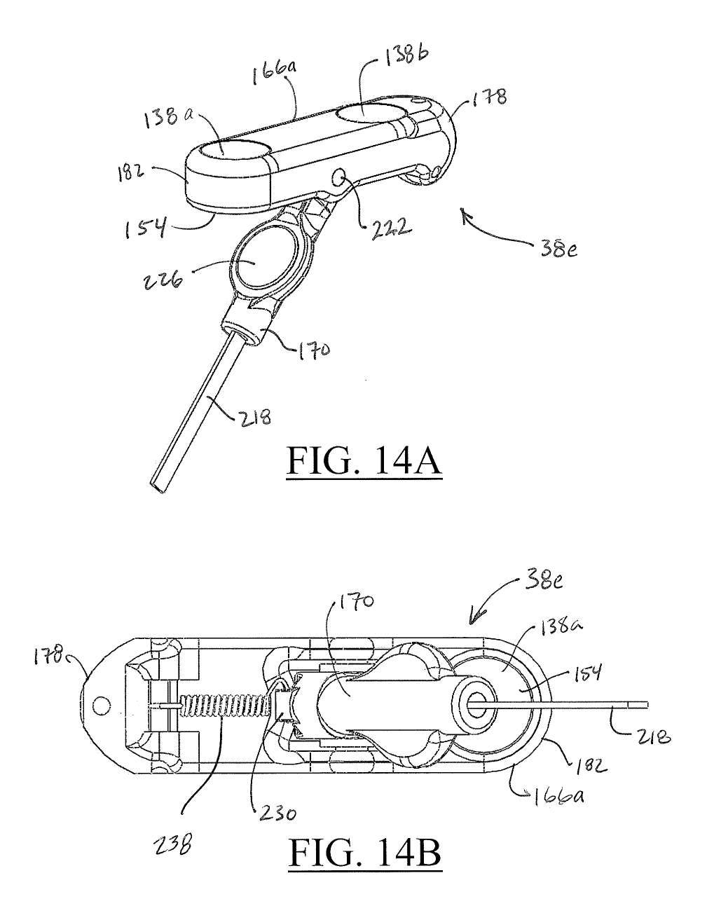

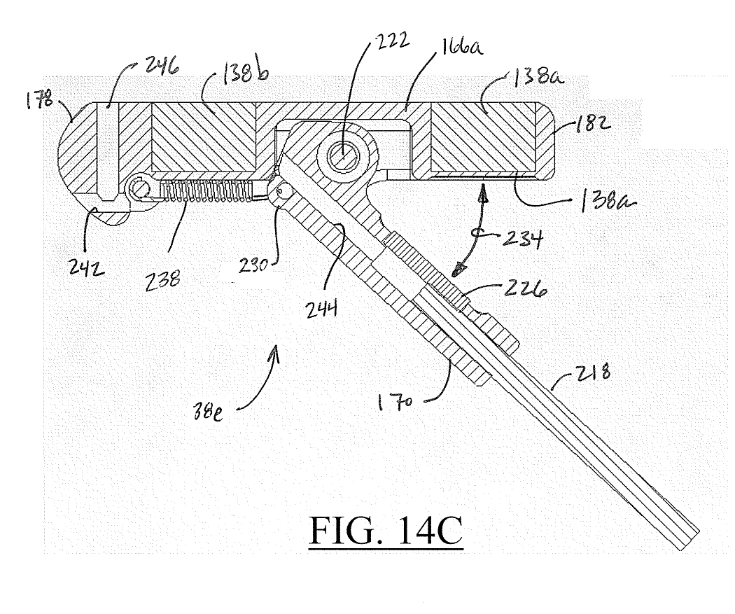

Referring now to FIGS. 14A-14C, various views are shown of medical device 38e, another embodiment of one of the present medical devices, which can also be used as part of one of the present systems. Medical device 38e is similar in several respects to medical device 38d of FIGS. 13A-13G, so generally only the differences between them will be described here. The version of arm 170 that is part of device 38e is pivotally coupled to platform 166a by a pin or axle 222, such that arm 170 is movable between (1) a collapsed position where distal end 198 of arm 170 is adjacent to platform 166a, or where arm 170 is substantially parallel to platform 166a, as shown in FIG. 13C, and (2) an expanded position where distal end 190 of arm 170 is spaced apart from platform 166a, or where arm 170 is oriented at a non-zero angle to platform 166a. Additionally, as shown, arm 170 can include a magnetically-attractive member 226, and one or both of member 226 and member 138a can be adapted, for example, using the magnetic principles described above, such that at least when arm 170 is in the collapsed position, a magnetic attractive force arises between member 226 and member 138a.

Arm 170 can also include a lug or stop 230, as best shown in FIG. 14C, configured to contact or engage a portion of platform 166a so as to limit the range of motion of arm 170 relative to the platform. For example, lug 230 can be configured such that the angle 234 cannot exceed about any of: 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, or 90 degrees. Device 38e can also include a spring 238 to bias arm 170 toward the expanded position. In some embodiments, device 38e can be configured such that when arm 170 is in the collapsed position, the magnetic attractive force between member 138a of the platform and member 226 of the arm is (1) large enough that in the absence of an external force, the arm is held against the tension of the spring in the collapsed position, and (2) small enough that it can be overcome with the external force caused by bumping or jarring the arm against a surface (such as an organ or piece of tissue within the body cavity) such that the tension of the spring (e.g., spring 238) pulls the arm into the expanded position. For example, in some embodiments, the biasing force provided along the axis of the spring (when the arm is in the collapsed position) is larger than the magnetic attractive force between member 138a and member 226, e.g., at least or greater than about 105 percent, 110 percent, 115 percent, or any other suitable percentage or ratio that permits the device 38e to function as described above. Additionally, platform 166a can be provided with a tether port 242 and a set screw 246 to secure tether 42 relative to platform 166a. For example, set screw 246 can be loosened, tether 42 inserted or connected within tether port 242, and the set screw tightened to clamp or pinch a portion of the tether to prevent the tether from pulling away from the platform. The embodiment shown also includes a channel 244 extending through the arm 170, for example, to permit a conductor (not shown) to pass through the channel 244 to enable electrical communication with, and provide power to, the cautery 218.

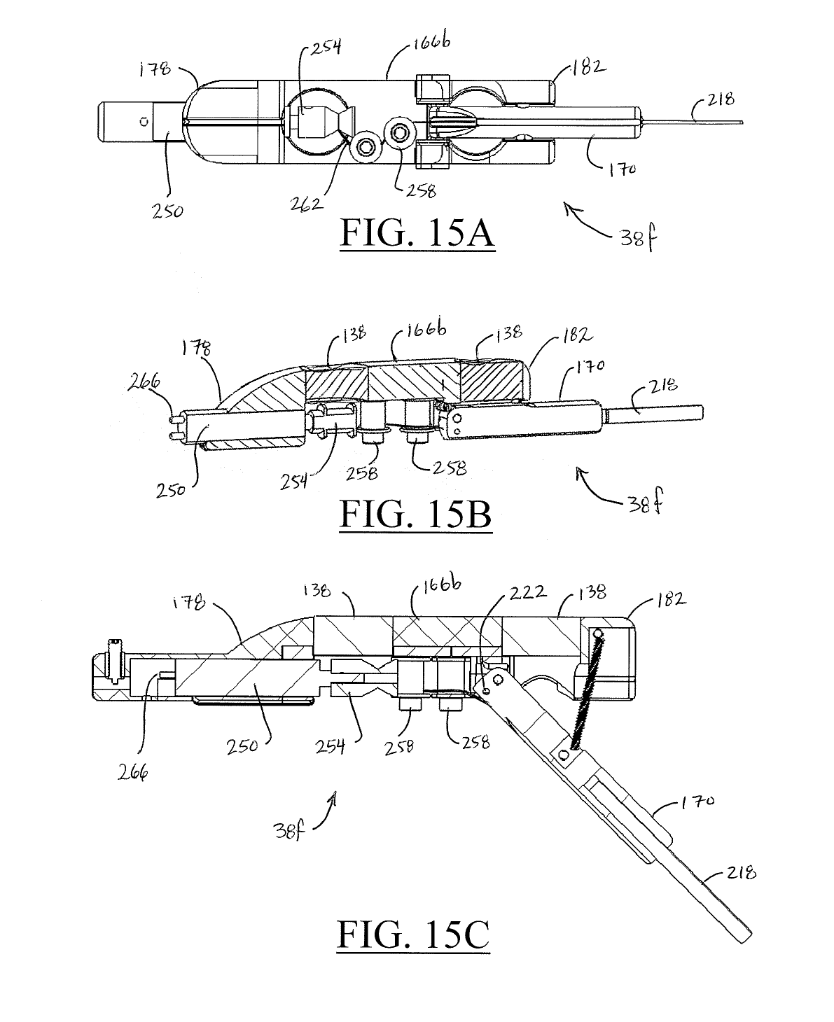

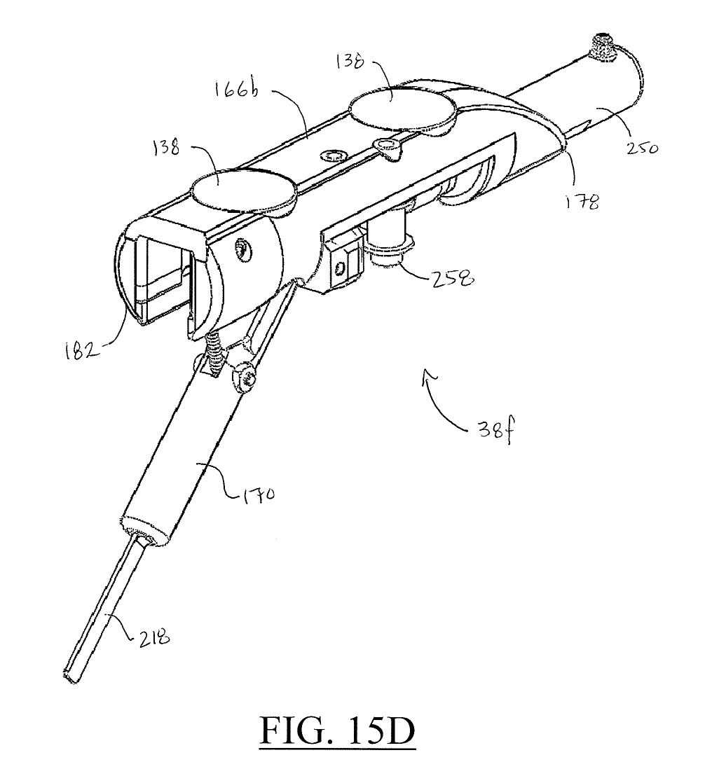

Referring now to FIGS. 15A-15D, various views are shown of medical device 38f, another embodiment of one of the present medical devices, which can also be used as part of one of the present systems. Medical device 38f is similar in several respects to medical devices 38e of FIGS. 14A-14C and 38d of FIGS. 13A-13G, so generally only the differences with these other embodiments of the present medical devices will be described here. Medical device 38f is configured such that arm 170 is pivotally coupled to platform 166b by a pin or axle 222. It can also be configured such that arm 170 can be biased toward the closed position by way of a spring or the like (not shown). Device 38f includes a motor 250 having a rotating output shaft or pulley 254, one or more pulleys 258 coupled to platform 166b, and a cord 262. As shown, cord 262 can connect to arm 170 at one end, pass about and in contact with a portion of each pulley 258, and connect to pulley 254 of motor 250 at the other end. In operation, motor 250 can be actuated to wind cord 262, thereby pulling arm 170 from the collapsed position to the expanded position; and motor 250 can be actuated to unwind cord 262, thereby releasing arm 250 to be drawn back into the collapsed position by the biasing spring (not shown, but see, e.g., spring 238 associated with device 38e). Motor 250 can be hydraulic or electric, and can include one or more connectors 266 to permit connection of a conduit or conductor, as described above, so that current or hydraulic fluid can be supplied to operate motor 250. For ease of illustration, magnetically-attractive members 138a, 138b are omitted from FIG. 15D.

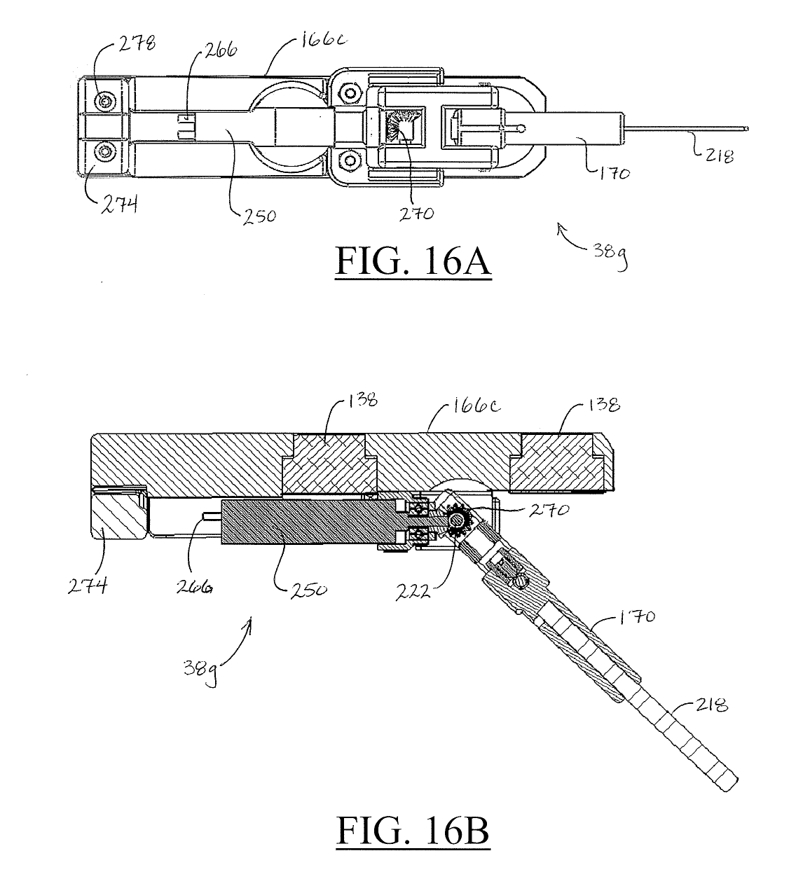

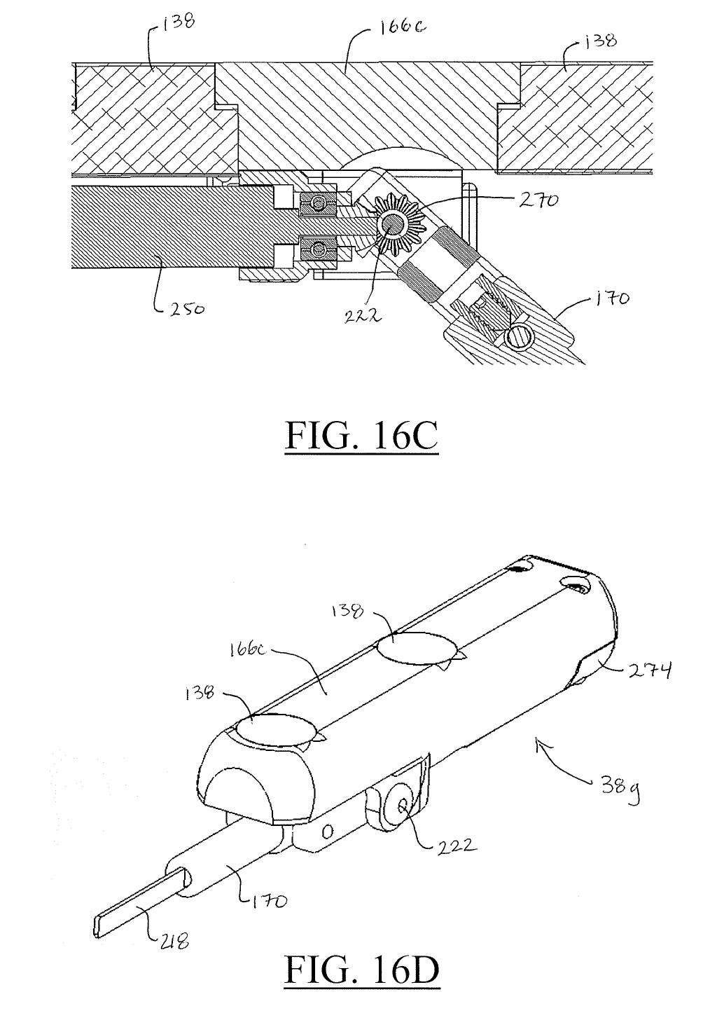

Referring now to FIGS. 16A-16D, various views are shown of medical device 38g, another embodiment of one of the present medical devices, which can also be used as part of one of the present systems. Medical device 38g is similar in several respects to medical devices 38f of FIGS. 15A-15D, 38e of FIGS. 14A-14C; and 38d of FIGS. 13A-13G, so generally only the differences with these other embodiments of the present medical devices will be described here. Medical device 38g is configured such that arm 170 is pivotally coupled to platform 166c by a pin or axle 222. Device 38g includes a motor 250 with connectors 266. Motor 250 and arm 170 can be coupled with bevel gears 270 which can take the form of (for example) 45-degree bevel gears. As a result, rotation produced by motor 250 can be converted to rotational motion of arm 170 such that arm 170 can be deployed from the collapsed position to an expanded position by actuating motor 250. Additionally, device 38g can include a clamping portion 274 that is coupled to platform 166c with, in this example, screws 278. By loosening screws 278, clamping portion 274 can be separated from platform 166c such that tether 42 can be inserted between them and connected to connector 266. The tether can then be clamped between clamping portion 274 and platform 166c by tightening screws 278 to, for example, provide strain relief for the tether.

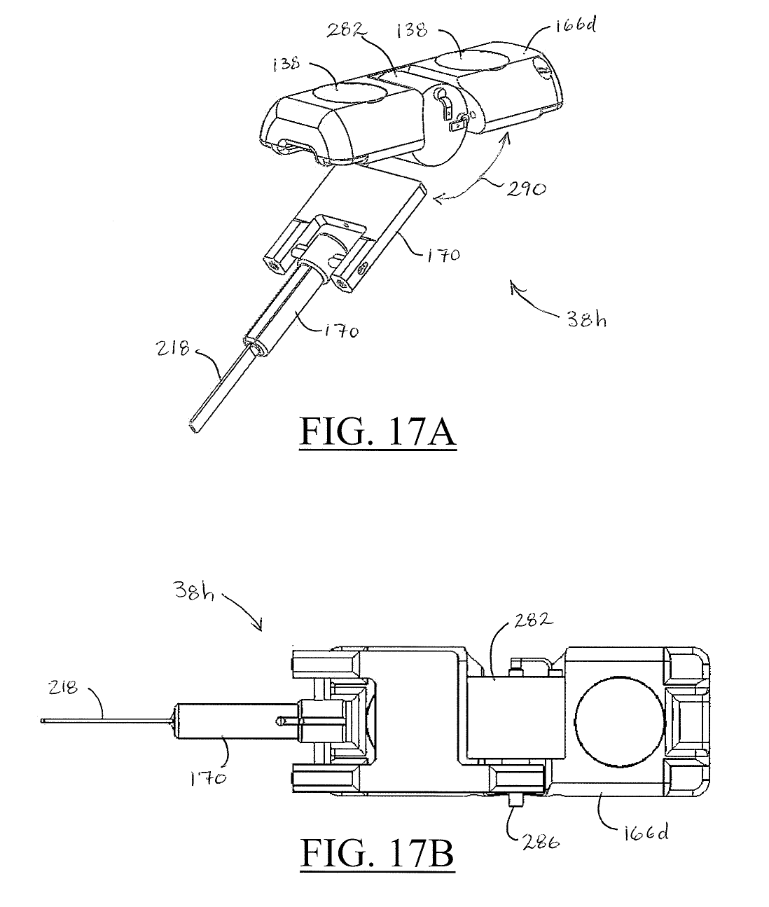

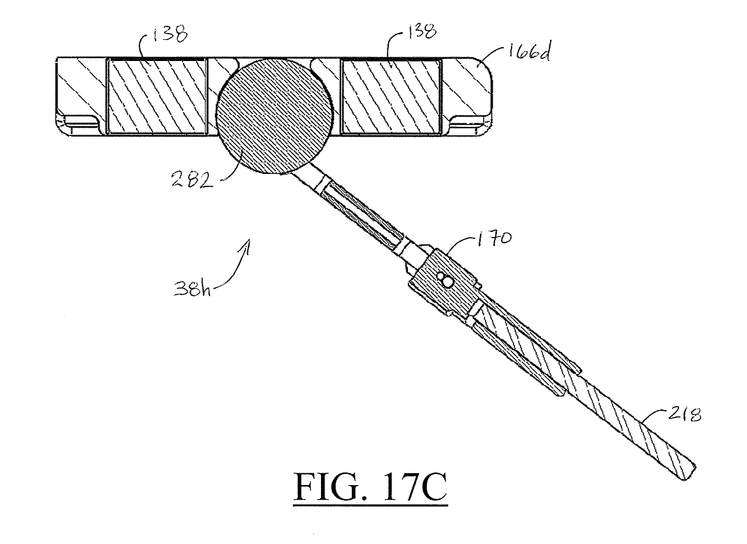

Referring now to FIGS. 17A-17C, various views are shown of medical device 38h, another embodiment of one of the present medical devices, which can also be used as part of one of the present systems. Medical device 38h is similar in several respects to medical devices 38g of FIGS. 16A-16D, 38f of FIGS. 15A-15D, 38e of FIGS. 14A-14C and 38d of FIGS. 13A-13G, so generally only the differences with these other embodiments of the present medical devices will be described here. Medical device 38h includes motor 282 with a shaft 286 that is perpendicular to the longitudinal axis of platform 166d. Arm 170 can be coupled to shaft 286 such that the rotation of the shaft translates directly into rotation of arm 170, allowing the arm to be deployed from the collapsed position to an expanded position. Other versions of device 38h include a gear reduction mechanism (not shown) that translates each revolution of shaft 286 into about any of: 5 degrees, 10 degrees, 15 degrees, 20 degrees, 30 degrees, 45 degrees, 90 degrees, 120 degrees, 180 degrees, 225 degrees, 270 degrees, or 315 degrees. Additionally, motor 282 can be configured such that the angle 290 between the collapsed position and the expanded position can be adjusted by actuating motor 282 to a desired degree. Examples of suitable motors for use as motor 282 include pancake gearhead motors, fluidic motors (both hydraulic and pneumatic), fluidic cylinder rack and pinion drives, ballscrews, and the like. In some embodiments, device 38h is configured such that when arm 170 is in a deployed position relative (e.g., at angle 290) to platform 166d, tip 218 is configured to be rotatable relative to arm 170. For example, in some embodiments, arm 170 can comprise a motor configured to rotate tip 218 relative to arm 170. In some embodiments, device 38h can be configured such that arm 170 is rotatable laterally relative to platform 166d. For example, in the embodiment shown, device 38h is configured such that arm 170 rotates in a vertical plane (around a horizontal axis) relative to platform 166d. The axis of rotation can be angled relative to platform 166d (e.g., at a 45-degree angle such that arm moves from its collapsed position to its deployed position along a path that moves arm 170 both vertically and laterally relative to platform 166d) such that, for example, the vertical displacement of tip 218 relative to platform 166d is reduced.

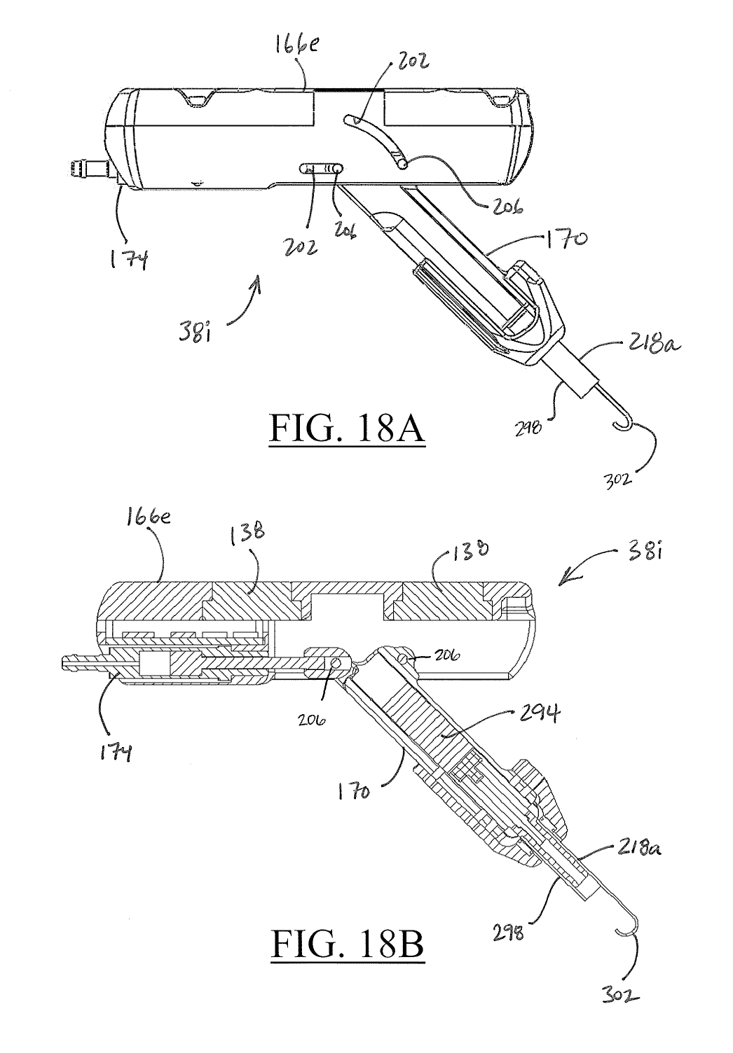

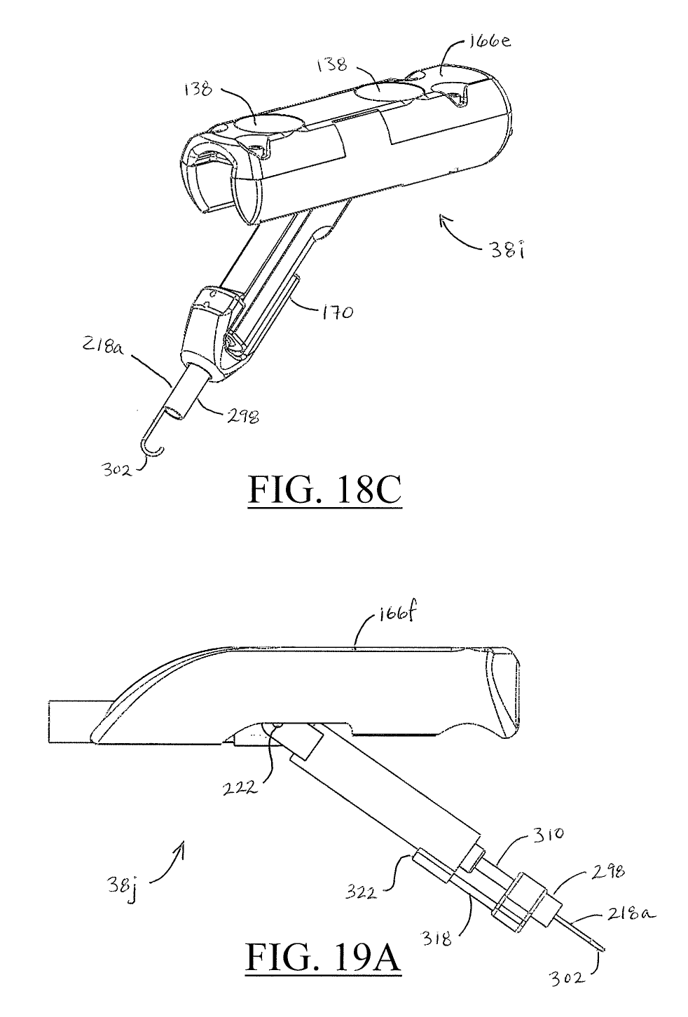

Referring now to FIGS. 18A-18C, various views are shown of medical device 38i, another embodiment of one of the present medical devices, which can also be used as part of one of the present systems. Medical device 38i is similar in several respects to medical devices 38h of FIGS. 17A-17C, 38g of FIGS. 16A-16D, 38f of FIGS. 15A-15D, 38e of FIGS. 14A-14C and 38d of FIGS. 13A-13G, so generally only the differences with these other embodiments of the present medical devices will be described here. As with device 38d shown in FIGS. 13A-13G, arm 170 is coupled to platform 166e by way of cams slots 202 and pins 206, and can be actuated between collapsed and expanded positions by cylinder 174. Additionally, device 38i includes a motor 294 coupled to tool 218a that is configured to cause the tool to rotate about its longitudinal axis when the motor is actuated. Examples of suitable motors for use as motor 294 include electric motors, hydraulic motors, and ceramic motors. In the embodiment shown, tool 218a is a cautery tool that includes a base portion 298 and a hook portion 302 that comprises an electrode surface that can be energized (e.g., a “working” surface). Thus, the cautery tool is configured such that, when activated, it can perform cutting and/or cauterizing functions (some of which may be intricate depending on the size of the hook), and, when not activated, it can be used to pull or push items such as tissue and organs, and in some cases sutures, blood vessels, and the like.

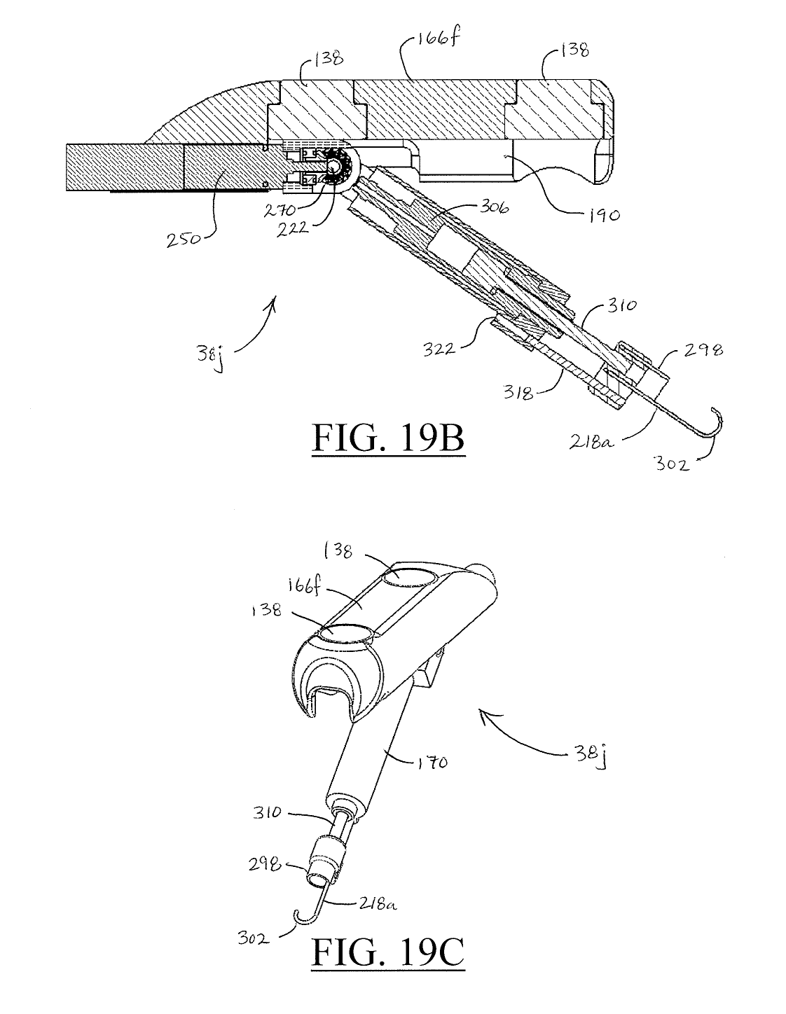

Referring now to FIGS. 19A-19C, various views are shown of medical device 38j, another embodiment of one of the present medical devices, which can also be used as part of one of the present systems. Medical device 38j is similar in several respects to medical devices 38i of FIGS. 18A-18C, 38h of FIGS. 17A-17C, 38g of FIGS. 16A-16D, 38f of FIGS. 15A-15D, 38e of FIGS. 14A-14C and 38d of FIGS. 13A-13G, so generally only the differences with these other embodiments of the present medical devices will be described here. As with device 38g shown in FIGS. 16A-16D, arm 170 is pivotally coupled to platform 166f by way of pin or axle 222, and can be actuated between collapsed and expanded positions by motor 250 and bevel gears 270. Additionally, device 38j includes a cylinder 306 that has a piston 310 and an inlet 314. Piston 310 can be coupled to cautery tool 218a such that the cautery tool can be extended outward (e.g., from an non-extended position to an extended position) along its longitudinal axis by actuating cylinder 306. Inlet 314 can be coupled to a fluid conduit of tether 42 as discussed above by way, for example, of a port or conduit (not shown) that is attached to or defined within platform 166f. Additionally, device 38j can include a guide rod 318 that is coupled to piston 310 and/or cautery tool 218a and positioned in (and slidable within) guide passageway 322 that is parallel to the piston and defined within a portion of arm 170. Guide rod 318 and/or passageway 322 can be configured to prevent piston 310 from rotating relative to cylinder 306, or to prevent piston 310 from extending beyond a predetermined position relative to cylinder 306.

Embodiments of the present medical devices (e.g., 38, 38a, 38b, 38c, 38d, 38e, 38f, 38g, 38h, 38i, 38j and 38k) can be made by any suitable method and can comprise any suitable material or materials. For example, the platforms (e.g., 166, 166a, 166b, etc.) and arms 170 can be machined by conventional subtractive methods such as milling or turning, or can be formed by additive methods such as those used for rapid prototyping; and can comprise suitable biocompatible materials such as plastics, metals, composites, alloys, and the like. Various other components such as, for example, bearings, gears, fluid cylinders, cables, conductors, conduits, and the like can be obtained from common mechanical/electrical suppliers, such as, for example, Small Parts, Inc., Florida, USA; McMaster-Carr Supply Company, Georgia, USA; Stock Drive Products/Sterling Instrument, New York, USA; SMC Corporation of America, Indiana, USA; Bimba Manufacturing Company, Illinois, USA; Festo Corporation, New York, USA; Faulhaber Group, Germany; and MicroMo Electronics, Inc., Florida, USA. Similarly, the parts or components of embodiments of the present systems and/or medical devices can be assembled through any suitable means including, for example, conventional manual techniques, fastening, press-fitting, securing with biocompatible epoxies or adhesives, and the like. In embodiments of the present systems and medical devices that include tether 42, and tether 42 serves to couple the tool of the device to a power source, the source can be a hydraulic source such as a fluid (liquid or gas) pressure source. Examples of fluid pressure sources include hand pumps, electric pumps, compressed gas bottles with a pressure regulator, or the like. In embodiments in which the power source that tether 42 can couple to the tool is an electrical power source, examples of such power sources include batteries, electric amplifiers, and the like. Other examples of electrical power sources that can be used where the tool is a cautery tool include, as mentioned above, electrosurgery units , such as, for example, an electrosurgery unit or power source available from suppliers such as, for example, ValleyLab, Colorado, USA; Erbe USA, Inc., Georgia, USA. In some embodiments, tether 42 can include more than one conductors and/or conduits. For example, tether 42 can include one conductor and one conduit, two conductors and one conduit, three conductors, or the like, as appropriate for delivering hydraulic fluid (gas or liquid) and/or electric power to various components of the relevant device (e.g., 38, 38a, 38b, 38c, 38d, 38e, 38f, 38g, 38h, 38i, 38j and 38k). By of way additional examples, the tether 42 can include a conductive portion coaxially about a fluid conduit, or can include a conductive portion (insulated) within a fluid conduit (e.g., configured to permit fluid to flow within the conduit adjacent to the conductor).

In embodiments of the present devices and systems configured such that arm 170 can be deployed by a user from the collapsed position to an expanded position using a motor (e.g. those embodiments that include devices 38e, 38f, or 38i), the motor can be controlled by a switch (not shown), such as, in some embodiments, a three-position switch (e.g., clockwise, neutral or off, and counter-clockwise). Similarly, embodiments of the present devices and systems configured such that arm 170 can be deployed by a user from the collapsed position to an expanded position using a cylinder (e.g., those embodiments that include devices 38d or 38h), the cylinder can be controlled by a switch that controls the hydraulic pressure source (not shown), such as, in some embodiments, a three-position switch (e.g., expansion or forward, neutral or locked, and contraction or reverse). In some embodiments, arm 170 or another portion of the device can be provided with a position sensor for sensing the position of the arm. Examples of suitable position sensors include potentiometers, limit switches, and encoders. In some of these embodiments, the position sensor can be configured to stop motion of the arm when the arm has reached a predetermined position.

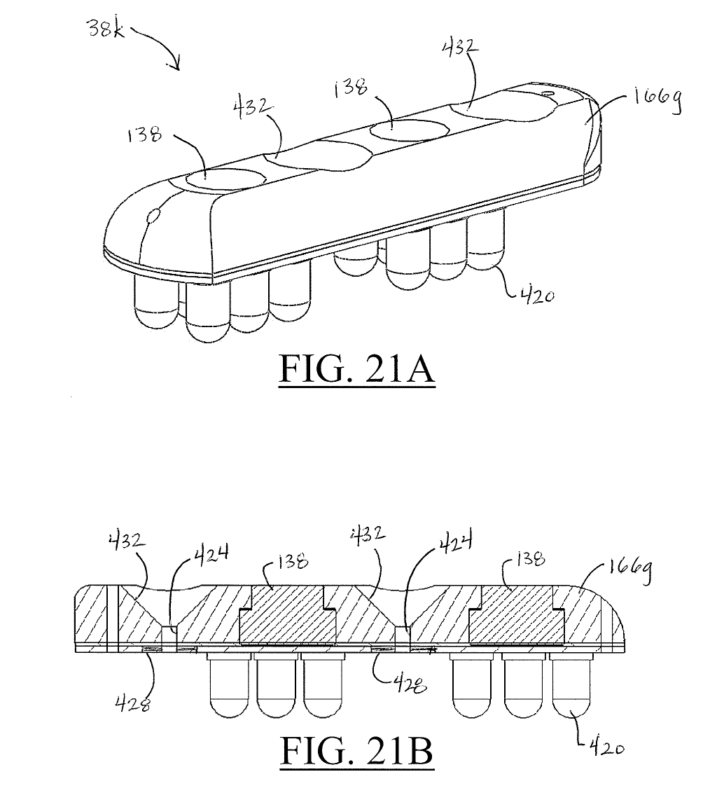

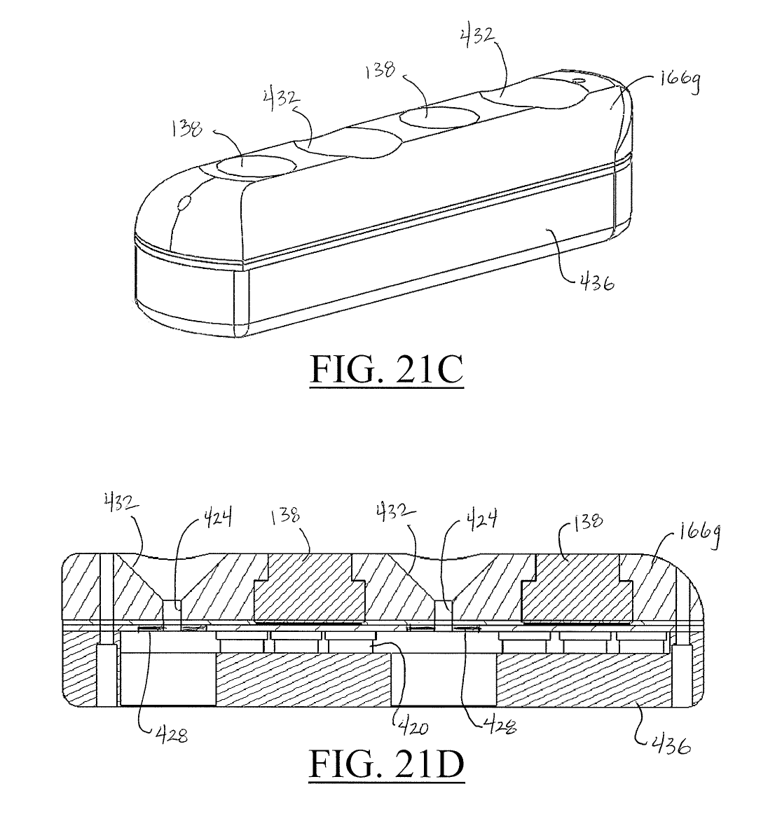

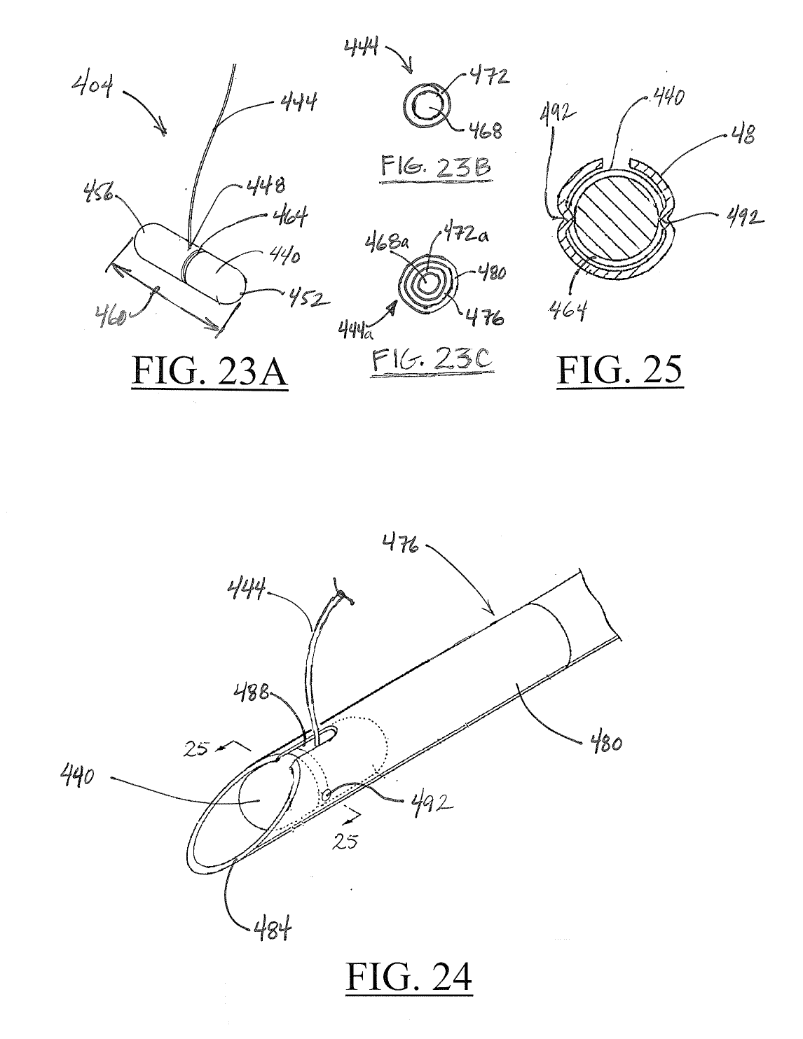

Referring now to FIG. 20, shown there and designated by reference numeral 400 is one embodiment of a system for enabling electrical communication with one of the present medical devices. System 400 comprises an apparatus for enabling electrical communication with device 38, though the system can be used with any of the devices described here (e.g., device 38k of FIGS. 21A-21D). More particularly, in the embodiment shown, the system comprises two apparatuses 404 (shown in more detail in FIGS. 23A-23C and described in more detail below) and two clamps or locks 408 for securing the apparatuses. As shown, each apparatus 404 extends through a puncture in the exterior surface 30 of wall 22, which can also be an exterior surface 30 of patient 14. Some versions of system 400 include a power source 412, which can include a positive connection 416a and a negative connection 416b. In some embodiments of the present systems of the type represented by system 400, the apparatus or apparatuses can be configured to enable electrical communication between it or them and the device (e.g., medical device 38), and to be connectable to the connections of the power source. Exemplary apparatuses 404 are described below with reference to FIGS. 23A-23C. For example, one of the two apparatuses 404 can be connectable to one of connections 416a or 416b, and the other of the two apparatuses 404 can be connectable to the other of connections 416a or 416b. In versions of system 400 that include a power source, the power source can comprise any suitable source of voltage or current and/or any suitable device or system for modifying the voltage or current from the source, such as, for example, an electrical outlet, a voltage converter, and AC/DC converter, a voltage regulator, or any suitable combination of these.

Referring now to FIGS. 21A-21D, various views are shown of medical device 38k, another embodiment of one of the present medical devices, which can also be used as part of one of the present systems. Medical device 38k is similar in several respects to medical devices 38j of FIGS. 19A-19C, 38i of FIGS. 18A-18C, 38h of FIGS. 17A-17C, 38g of FIGS. 16A-16D, 38f of FIGS. 15A-15D, 38e of FIGS. 14A-14C and 38d of FIGS. 13A-13G, so generally only the differences with these other embodiments of the present medical devices will be described here. Device 38k is shown without an arm 170 or any corresponding structure for locomotion of arm 170. Instead, device 38k is shown with a plurality of light sources 420 that can be used, for example, to illuminate cavity 18 or a portion or point within the cavity in which the device is used. Light sources 420 can take any suitable form including, for example, light emitting diodes (LEDs). Device 38k can also be adapted for use with an apparatus (e.g., apparatuses 404) of, for example, system 400. For example, device 38k can be provided with a number of openings 424 that correspond to the number of apparatuses to be used with device 38k, such as, for example, one, two, three, or more openings 424. Additionally, device 38k can be provided with a conductive portion 428 for example, at or near working side 146 of device 38k. In some embodiments, a conductive portion 428 of device 38k can be adjacent to each opening 424; in other embodiments, a conductive portion 428 of device 38k can substantially surround an opening 424. Conductive portions 428 can each take any suitable form including, for example, silver, copper, silver-covered copper, or any other suitably conductive materials (e.g., metals, polymers). In some embodiments, conductive portions 428 each includes a groove (e.g., having co-linear portions opposite sides of opening 424) configured to receive a portion (e.g., anchor 440) of an apparatus 404, such as, for example, to resist rotation of apparatus 404 (and/or anchor 440) relative to device 38k when apparatus 404 is under tension. As a result of configuring device 38k in this manner, an apparatus 404 can pass through an opening 424 (which can also be described as a passageway 424) and contact an adjacent conductive portion 428 such that electrical communication is enabled between the apparatus and conductive portion 428. One of conductive portions 428 can be used for a positive connection and the other conductive portion can be used for a negative connection. Device 38k can also be configured such that light sources 420 are in electrical communication with conductive portions using, for example, wires, conductive traces, or a direct connection, such that when the apparatuses are connected to power supply 412 current is permitted to flow and energize the light sources to emit light. For example, some embodiments of device 38k can comprise a circuit board (e.g., a printed circuit board or PCB) comprising conductive traces electrically coupling the LEDs to conductive portions 428; and/or comprising circuitry configured to operate independent of polarity (e.g., the polarity of apparatuses 404) and/or configured to reverse polarity if the polarity of apparatuses 404 is reversed (e.g., from an intended or expected polarity). In some embodiments, apparatuses 404 are configured (e.g., via different sizes or shapes) to correspond to appropriate polarities. For example, in some embodiments, a positive apparatus 404 (apparatus configured for positive polarity) is configured to have a larger size and/or different shape than a negative apparatus 404 (apparatus configured for negative polarity). Additionally, device 38k can include an enlarged tapered portion 432 that, as shown, can have an inverted conical shape, at one end of each opening 424 so as to facilitate insertion of an apparatus (e.g., 404) into the opening.

As shown in FIGS. 21C and 21D, device 38k can also include a cover 436 that is coupled to platform 166g by any suitable device or structure, such as, for example, adhesive, clips, screws, rivets, bolts, or any other suitable means. The cover can be configured such that it permits at least some portion of the light emitted by light sources 420 to pass through the cover. In some embodiments, cover 436 can be configured to be substantially clear. The material used for cover 436 can be chosen so that it protects the interior of cavity 18 from being electrically shocked by conductive portions 428, and prevents any portion of the apparatuses from falling into the cavity 18. The cover can comprise, for example, clear or translucent polycarbonate (e.g., LEXAN brand polycarbonate resin thermoplastic), or the like. In some embodiments, the cover is resistant to shattering, non-conductive, and/or capable of being sterilized.

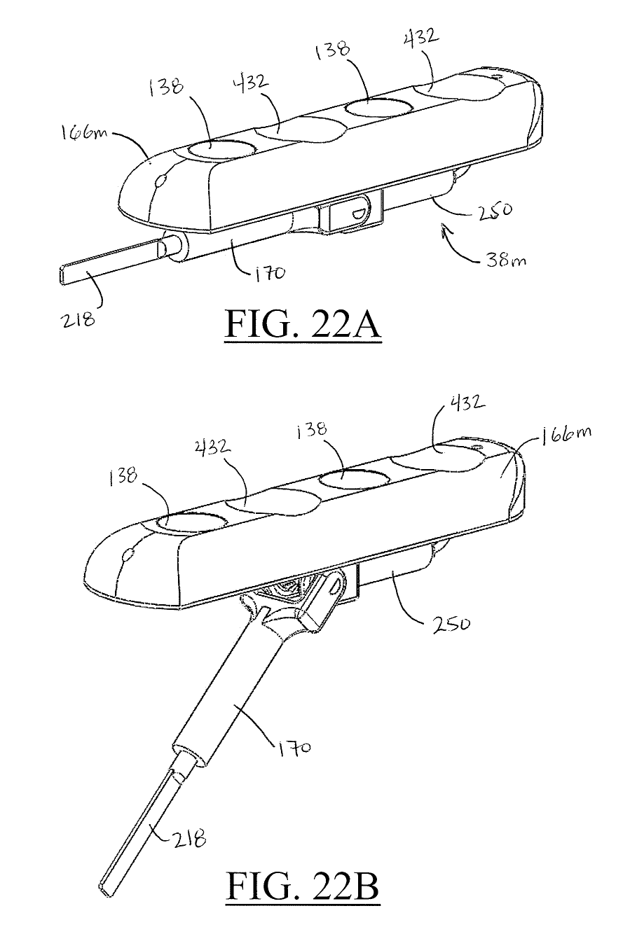

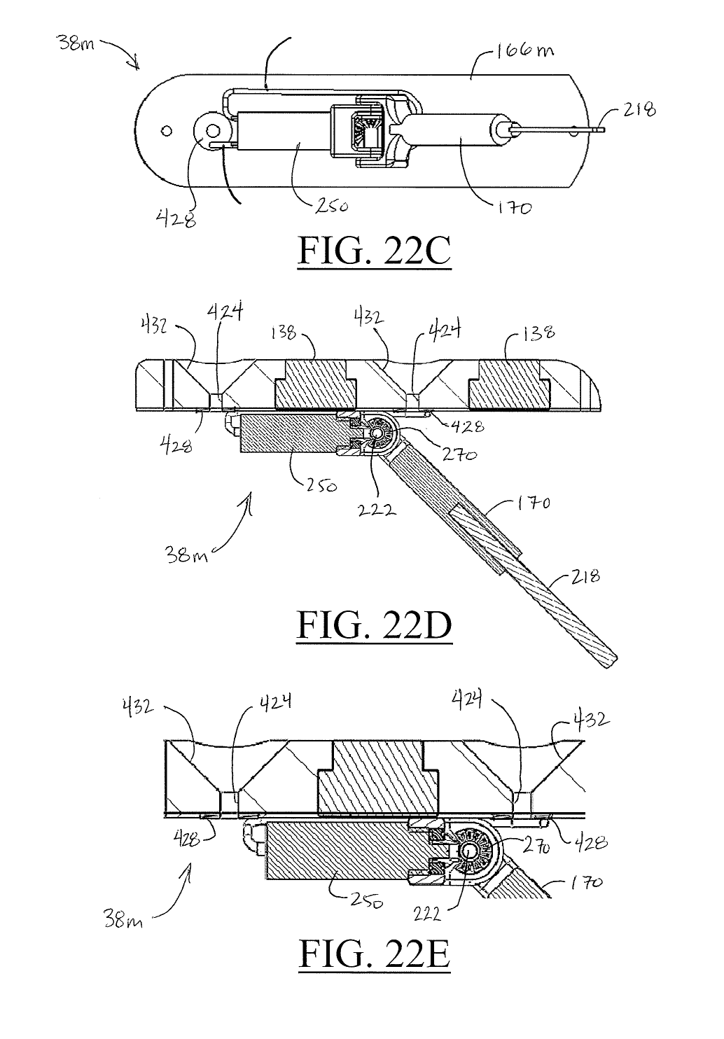

Referring now to FIGS. 22A-22E, various views are shown of medical device 38m, another embodiment of one of the present medical devices, which can also be used as part of one of the present systems. Medical device 38m is similar in several respects to medical devices 38k of FIGS. 21A-21D, 38j of FIGS. 19A-19C, 38i of FIGS. 18A-18C, 38h of FIGS. 17A-17C, 38g of FIGS. 16A-16D, 38f of FIGS. 15A-15D, 38e of FIGS. 14A-14C and 38d of FIGS. 13A-13G, so generally only the differences with these other embodiments of the present medical devices will be described here. Motor 250 and gears 270 are similar to those of device 38g of FIGS. 16A-16D. However, openings 424, conductive portions 428, and tapered portions 432 are similar to those of device 38k of FIGS. 21A-21D. Additionally, device 38m is shown with connectors 438 extending between, and in electrical communication with, conductive portions 428 and motor 250, such that device 38m is configured to function with apparatus 404 for enabling electrical communication, as described in this disclosure. Connectors 438 can comprise any suitable conductive connectors, such as, for example, conductive wire (insulated or uninsulated), conductive connectors integrally formed with conductive portions 428, or any other suitable connector.

Referring now to FIG. 23A, a perspective view is shown of apparatus 404. As shown, apparatus 404 can comprise an anchor 440 and a conductor 444 connected to anchor 440 at a connection point 448. The anchor can have a first end 452, a second end 456, and a length 460 extending between first end 452 and second end 456. In some embodiments, connection point 448 can be about midway between first and second ends 452 and 456. Anchor 440 can also include a recess 464 extending about (or, when the anchor has a cylinder-like shape as shown, circumscribing) a portion of anchor 440, or all the way about anchor 440, as described in more detail below. In some embodiments, anchor 440 can comprise a tube and/or tubular (hollow) cross-section; and/or can comprise stainless steel (e.g., a stainless steel tube having length 460). Anchor 440 and conductor 444 can be coupled to one another in any suitable fashion (e.g., crimping and/or soldering).

Referring now to FIGS. 23B and 23C, cross-sectional views of two embodiments of conductor 444 are shown. In the first embodiment shown in FIG. 23B, conductor 444 comprises a first or central conductive portion 468 and an outer layer of insulating material 472 disposed about the conductive portion 468. These two portions can be substantially coaxial, as shown. In some embodiments, conductor 444 comprises magnet wire (e.g., central conductive portion 468 can comprise copper and/or insulating material 472 can comprise enamel). In the second embodiment shown in FIG. 23C, conductor 444a comprises a first or central conductive portion 468a, a first layer of insulating material 472a disposed about the first conductive portion 468a, a second conductive portion 476 disposed about the first layer of insulating material 472, and a second or outer layer of insulating material 480 disposed about the second conductive portion 472. These four portions can be substantially coaxial, as shown.

In FIG. 24, a perspective view is shown of a deployment needle 476 for deploying apparatus 404. In some embodiments, needle 476 comprises an 18-gauge needle (e.g., apparatus 404 is configured to fit in and/or be delivered by an 18-gauge needle). FIG. 25 shows a cross-sectional view, taken along line 25-25 in FIG. 24, of deployment needle 476 and anchor 440 disposed within it. Deployment needle 476 can also be referred to as, simply, needle 476. Needle 476 can be configured similarly to a hypodermic needle; thus, and for example, needle 476 can comprise a hollow tubular body 480 and a tip 484 that can be angled and sharpened to facilitate insertion through tissue, such as external surface 30 of patient 14. As shown, needle 476 can be sized to receive a at least a portion of (up to all of) anchor 440 of apparatus 404. In some embodiments, needle 476 can also include a longitudinal slot 488 defined in body 480 that supports conductor 444 in relation to anchor 440 when anchor 440 is disposed within the needle as shown. In other embodiments, needle 476 may be configured to permit the conductor to extend through the hollow portion of the needle when a portion or all of anchor 440 is disposed within the needle. Body 480 can also include one or more (e.g., two) dimples or protrusions 492 positioned to correspond to recess 464 in anchor 440 when the anchor is disposed within needle 476. Dimples 492 can extend into recess 464 so as to prevent anchor 440 from falling out of the needle unless a force is applied to the anchor. In some embodiments, the dimples can be omitted such that anchor 440 can freely slide into and out of needle 476.

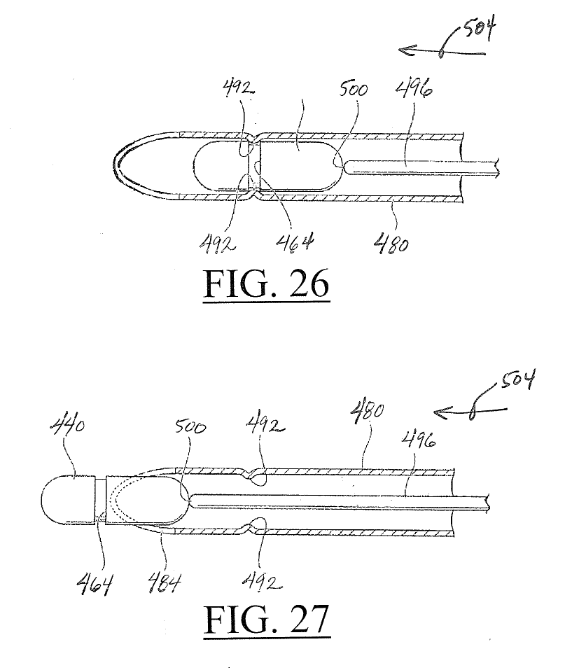

Referring now to FIGS. 26 and 27, two cross-sectional views are shown of needle 476 in which anchor 440 is disposed. For clarity, conductor 444 has been omitted. Needle 476, or an apparatus used in conjunction with needle 476, can comprise a pushrod 496 with an end 500 that extends through the hollow portion of body 480, as shown. When anchor 440 is “seated” in this embodiment of needle 476 (e.g., when anchor 440 is positioned within the needle such that dimples 492 of needle 476 extend into recess 464 of anchor 440), end 500 of pushrod 496 can be configured to be near or in contact with anchor 440 such that when it is desirable to deploy anchor 440 from needle 476, the pushrod can be pushed a short distance in direction 504 to deploy or eject the anchor from the needle. As shown in FIG. 27, pushrod 476 can be configured so that it is long enough, and movable a sufficient distance, that anchor 440 can be pushed beyond dimples 492 and far enough in direction 504 that the anchor will exit the needle.

In other embodiments, device 38k (and/or other embodiments of the present devices) can be configured to be coupled to needle 476 (with or without apparatus 404). For example, openings 424 can comprise female threads, and/or needle 476 can comprise male threads, such that needles 476 can be threaded into openings 424 to provide mechanical and/or electrical connections to the device.

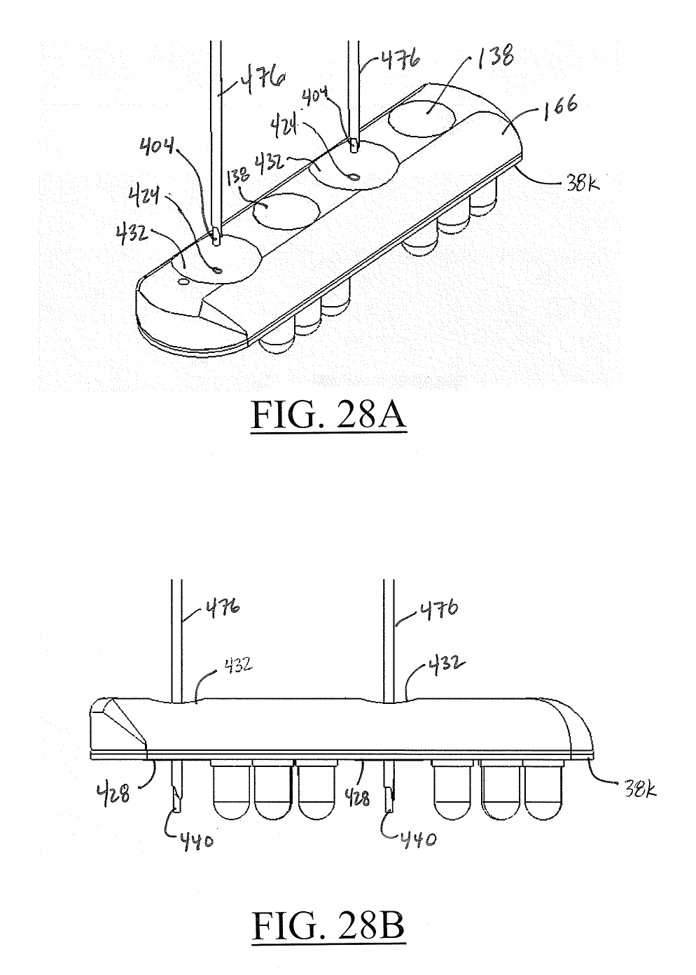

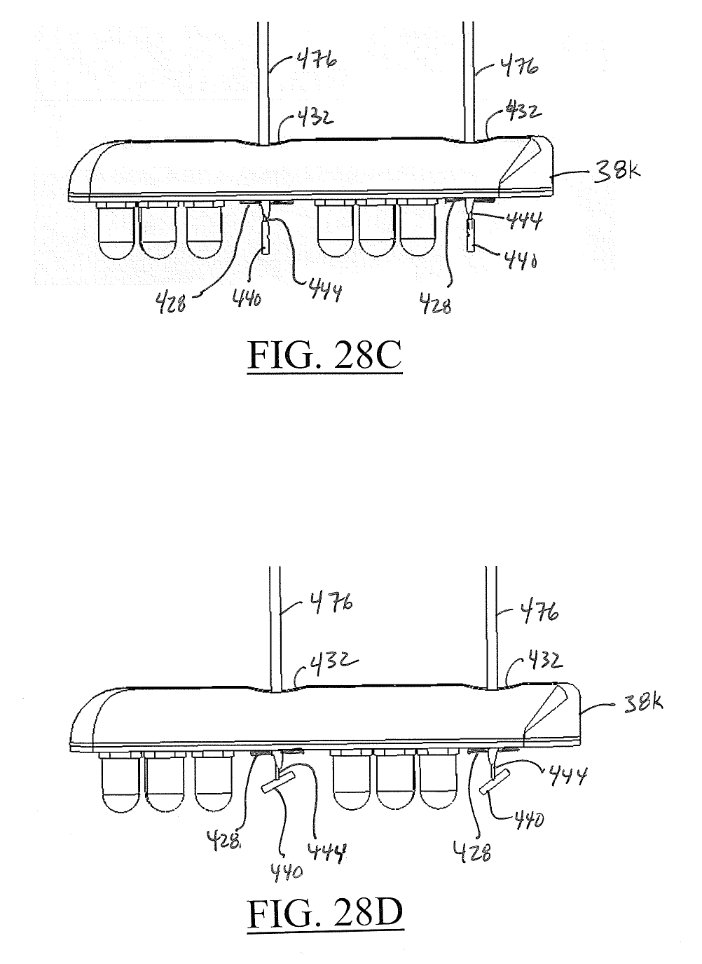

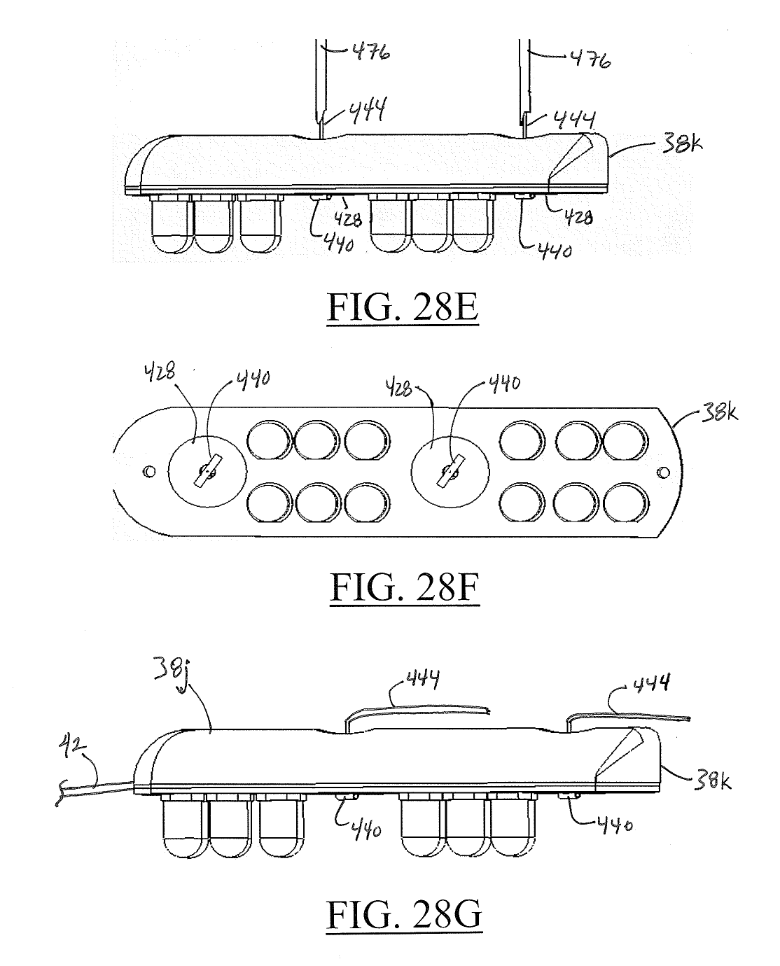

Referring now to FIGS. 28A-28G, various views are shown depicting various stages or steps of an apparatus 404 being deployed relative to device 38k, one of the present medical devices. For clarity, patient 14, cavity 18, and wall 22 are not shown in FIGS. 28A-28G; however, it should be understood that apparatus 404 can be deployed relative to one of the present medical devices (such as device 38k) positioned in a cavity of a patient (as shown in FIG. 20). For example, any of the present devices can be introduced into a cavity 18 via an access port (e.g., a trans-abdominal access port, trans-gastric access port, or NOTES access-port) and directed, driven, and/or translated to an appropriate location in the cavity with an apparatus (e.g., apparatus 38 of FIG. 1). Also for clarity, device 38k is shown without cover 436 in these figures; however, it should be understood that the method described can be used with a version of device 38k that includes cover 436. In some embodiments of the present systems, guide holes 66 of apparatus 34 (FIG. 3A) can be configured to align with holes 424 of device 38k when the apparatus and the device are magnetically coupled, such that apparatuses 404 can be deployed while the apparatus and the device are magnetically coupled. As a result, once the apparatuses 404 are deployed, they can secure or support device 38k such that apparatus 34 can be removed.

In the embodiments shown, two needles 476 can be used to insert apparatuses 404 into each of two openings 424 of device 38k. Although two needles 476 are shown deploying the apparatuses simultaneously, in some embodiments, two needles 476 can be used to deploy the apparatuses, sequentially; or one needle 476 can be used to deploy a single apparatus 404, or more than two apparatuses sequentially. As such, the deployment of one needle 476 deploying one apparatus 404 is described.

Once device 38k is moved to or disposed in a desired position with, for example, an apparatus 34, deployment needle 476 having at least anchor 440 of an apparatus 404a disposed within it can be used to insert the anchor and a portion of conductor 444 into an opening 424 of the device. In the embodiment shown, anchor 440 of apparatus 404a is partially disposed within needle 476 and conductor 444 extends through the hollow portion of body 480 of needle 476. Needle 476 can be located in a position (e.g., outside wall 22) where it is substantially aligned with at least a portion of opening 424 (e.g., enlarged tapered portion 432), as shown in FIG. 28A. Needle 476 can then puncture the exterior surface of the cavity wall, or can be inserted through a pre-formed puncture in the cavity wall. As shown in FIG. 28B, needle 476 can then be inserted into opening 424 and, in the embodiment shown, through device 38k. As the needle passes through the opening or after the end of the needle has passed through the opening, anchor 440 can be permitted to begin to exit the needle by, for example, pushing anchor 440 with pushrod 496 (FIGS. 26 and 27), by using conductor 444 to push anchor 440 from needle 476, or by allowing enough slack in conductor 444 to permit anchor 440 to exit needle 476.

As shown in FIG. 28C, anchor 440 is preferably pushed or permitted to completely exit needle 476. Apparatus 404a can be configured such that, as anchor 440 clears needle 476, the anchor is permitted to, or is biased to, shift or rotate relative to conductor 444, as shown in FIG. 28D, such that the anchor becomes oriented at a non-zero angle relative to conductor 444. As shown in FIGS. 28E and 28F, conductor 444 can then be partially refracted such that the anchor contacts conductive portion 428 of device 38k to, for example, enable electrical communication between conductor 444 and conductive portion 428, and in some embodiments, between the anchor 440 and conductive portion 428. Additionally, in some embodiments, portions of anchor 440 that do not contact conductive portion 428 can be electrically insulated from the conductive portion by, for example, covering such portions of the anchor with an insulating material. In other embodiments, conductive portion 428 may be disposed within opening 428 such that anchor 440 comprises a non-conductive material, or can be entirely covered with an electrically-insulating material.

Additionally, in some embodiments, needle 476 can be retracted from device 38k, as shown in FIG. 28E. Following the removal of needle 476, conductor 444 can be connected to power source 412, directly or indirectly, such as by way of a plug or other connector (not shown) to enable electrical communication between the power source and conductor 444 such that electrical communication is enabled between power source 412 and conductive portion 428 of device 38k.

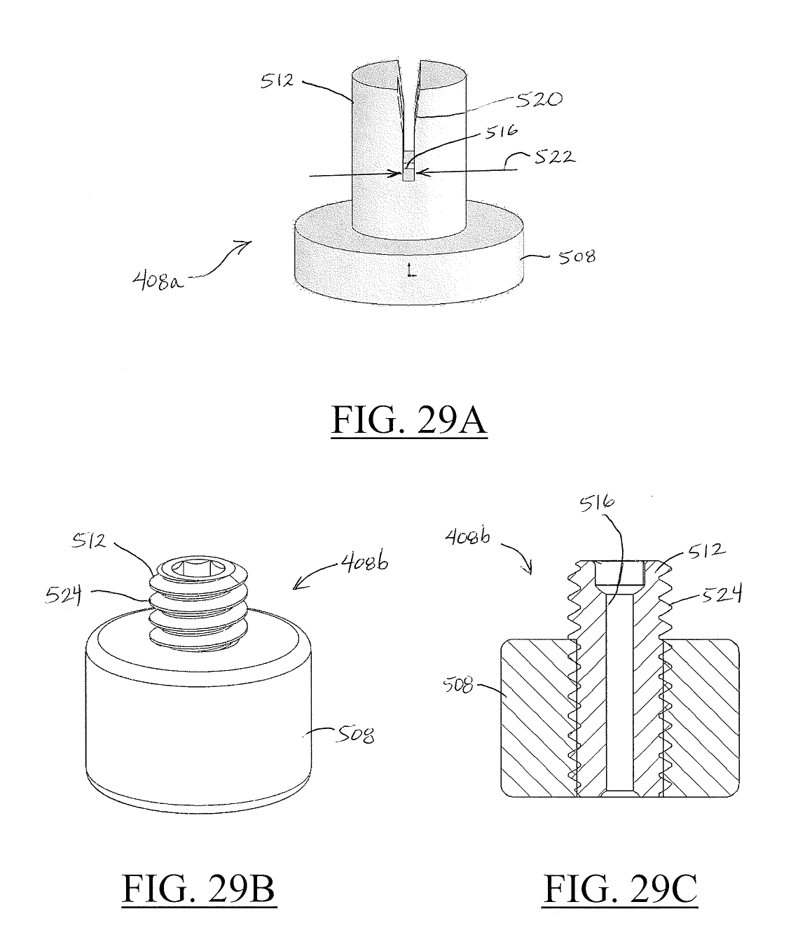

In some embodiments, apparatus 404a can be used to secure or support device 38k. For example, conductor 444 can be retracted enough that tension in the conductor holds device 38k against an interior surface of the cavity wall. Additionally, such a tension can be maintained in the conductor 444 by placing a lock 408 relative to an external surface (e.g., external surface 30) and conductor 444 (see FIG. 20) such that conductor 444, lock 408, and the exterior surface cooperate, directly or indirectly, to maintain the tension in the conductor and hold device 38k against the interior surface of the cavity wall. In some embodiments, lock 408 can directly contact the exterior surface, and in other embodiments, the lock may be indirectly supported by the exterior surface. The lock can be any suitable device for maintaining tension in conductor 444 as described, such as, for example, the version of lock 408 described below with reference to FIGS. 29A and 29B, or any other clamp, hemostat, or the like now known in the art or developed in the future.

To remove device 38k, lock 408 can be removed or disengaged from conductor 444. In some embodiments, the portion of conductor 444 outside the cavity wall can be pulled through the wall into the cavity (e.g., with graspers delivered and/or supported by one or more platforms, such as any of those described herein). In other embodiments, the portion of conductor 444 outside the cavity wall can be cut or trimmed off at a point outside the wall, and the remaining portion of conductor 444 can be pulled through the cavity wall into the body cavity. In such embodiments, device 38k can be pulled from the body cavity by way of tether 42 such that apparatus 404 is pulled with the device (e.g., such that at least a portion of apparatus 404 is trapped or sandwiched between the device and the peritoneum of the body cavity), as shown in FIG. 28G. Additionally, in versions of device 38k that include cover 436, the cover can be configured to catch anchor 440 and/or conductor 444 if it falls through opening 424 so as to prevent the anchor and/or the conductor from falling into the body cavity. In other embodiments, the foregoing steps and/or stages, described primarily with reference to FIGS. 28A-28G, can be reversed to remove conductor 444 and anchor 440 by way of the puncture through which they are inserted.

Referring now to FIGS. 29A and 29B, perspective views are shown of embodiments of locks 408 (408a and 408b) for use with the various embodiments of the present systems. In general, the locks can be used in conjunction with embodiments of apparatus 404 to hold or support the apparatus and/or to maintain tension in its conductor 444, as described above. In the embodiment shown in FIG. 29A, lock 408a includes an enlarged base portion 508 and an upper portion (or shaft) 512 that is defined by a smaller perimeter than is the base portion. Lock 408a also includes an opening 516 that extends from the end of the shaft into a least a portion of lock 408a, and in some embodiments, through the center of the entire lock 408a. Lock 408a can also include a slot 520 (as shown) that extends from the end of the shaft at least partially into the shaft. Slot 520a can bisect opening 516 and can also have a width 522 that is smaller than the diameter of conductor 444 and/or an enlarged tapered portion at an upper portion of slot 520, as shown. Conductor 444 can extend through opening 516 and be angled to extend from opening 516 through slot 520. In some embodiments, conductor 444 can be secured to the lock by wrapping the conductor about at least a portion of upper portion 512 of lock 408a. In embodiments where slot 520 has a width less than the diameter of the conductor, the conductor can be secured to the lock by positioning a portion of the conductor in the slot such that the conductor is pinched within the slot.

Lock 408b depicted in FIG. 29B is similar in some respects to lock 408a shown in FIG. 29A, so generally only the differences between the two are described here. The embodiment of lock 408b shown does not include a slot. Additionally, lock 408b includes a securing texture 524 about upper portion 512. Securing texture 524 can be configured to be capable of mechanically interacting with conductor 444 to hold or support conductor 444 in substantially fixed relation relative to lock 408b. In the embodiment shown, securing texture 524 comprises threads, as shown. In other embodiment, securing texture 524 can comprise, or be provided by, any suitable structure or configuration. For example, securing texture 524 can be provided by a helical spring positioned about upper portion 512. Conductor 444 can extend through opening 516 and be secured to the lock by wrapping the conductor about upper portion 512 such that that conductor engages and/or otherwise contacts securing texture 524. Other embodiments of lock 408b can include a slot and a securing texture.

In other embodiments, device 38k (and/or other embodiments of the present devices) can be configured to include one or more pins with holes or eyelets through which a hook or similar apparatus can be passed. For example, instead of openings 424, other embodiments of device 38k can comprise posts or pins (e.g., in tapered portion 432) each having a hole extending through the post (e.g., transverse to the longitudinal axis of the post) such that a wire, hook, or wire having a hook at its end, can be passed through the abdominal wall and inserted through the hole in the post on the device, to secure and/or power the device in a manner similar to that described above with reference to FIGS. 28A-28G.

In other embodiments, device 38k (and/or other embodiments of the present devices) can be configured to be powered through radio-frequency (RF) induction. For example, the device can comprise one or more conductive coils coupled to LEDs or the like (and/or a battery or the like configured to store energy); and/or an external apparatus (e.g., apparatus 34 of FIG. 1) can comprise one or more coils coupled to a power source; such that the one or more coils of the external apparatus can wirelessly couple to the one or more coils of the device to wirelessly (e.g., without wires extending between the external apparatus and the device) power the device.

In some embodiments, the motors, hydraulic cylinders, and/or other actuators can be substituted with, and/or supplemented by, one or more manual drives (e.g., a pull string or manual screw drive to advance and/or withdraw the arm and/or tip, a knob or the like configured to rotate a threaded rod in the arm such that a nut or the like coupled to the threaded rod can be linearly advanced and/or withdrawn by rotating the knob, and/or a knob configured to rotate the tip itself); one or more torsion springs configured to bias and/or hold the arm in a biased direction relative to the platform (e.g., collapsed or deployed); one or more linear compression springs configured to bias or hold the arm in a biased direction relative to the platform (e.g., configured to bias the arm open relative to the body such that when the arm is released the spring will deploy the arm to a deployed or open position relative to the platform); one or more fluid actuators (e.g., hydraulic cylinders, bladders, fluidic muscles such as tubes that will retract or extend with pressure, bellows, and/or fluidic rotary actuators such as those that can convert rotary motion to linear motion); and/or one or more electric or electromagnetic actuators (e.g., linear voice coils, piezoelectric actuators, rotary or gear motors such as those in which rotary motion is converted to linear motion, linear actuators, shape-memory alloys such as nickel-titanium (e.g., nitinol), and/or electro-active polymers that can be configured to change shape in the presence of an electrical field. Examples of piezoelectric actuators include: what may be known in the art as a “squiggle” in which a screw or bolt is vibrated through a nut; what may be known in the art as a “finger” that “flicks” or impacts a ceramic surface to cause motion; and/or the like. In one example of any embodiment of the present devices using shape-memory alloys and/or electro-active polymers, an alternate embodiment of device 38f can comprise a shape memory alloy and/or electro-active polymer in place of the reels and motor, such that the shape memory alloy and/or electro-active polymer can be configured to shorten and/or lengthen with the application of a voltage and/or current such that the arm can be deployed and/or collapsed. Any of the various actuators can be incorporated into any of the various embodiments of the present devices to actuate the arm relative to the body and/or the tip relative to the body (and/or the rest of the arm).

In any of the various embodiments described or suggested in this disclosure, the systems, apparatuses, devices, and methods can comprise or be limited to any combination of the features or characteristics that have been described, unless the context explicitly or necessarily precludes the combination. For example, an embodiment of one of the present devices (e.g., devices 38a, 38b, etc.) can comprise a platform (e.g. 166a, 166b, etc.) and an arm 170; another embodiment can comprise a platform, an arm, and a magnetically-attractive member 138; and another embodiment can comprise a platform and two magnetically-attractive members. As another example, an embodiment of system 400 (for enabling electrical communication with a device) can comprise an apparatus 404 and a lock 408; another embodiment can comprise two apparatuses 404, two locks 408, and a device 38k; and another embodiment can comprise two apparatuses 404 and a device 38.

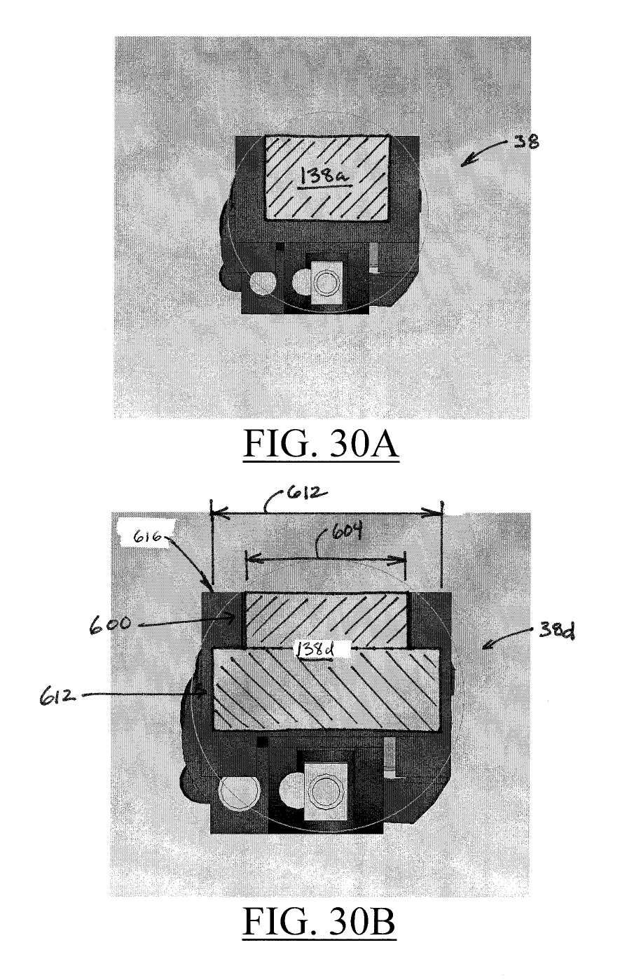

Referring now to FIGS. 30A and 30B, cross-sectional views of medical devices are shown illustrating two different configurations for magnets in the present medical devices. More particularly, FIG. 30A depicts a cross-sectional view of device 38e of FIGS. 14A-14C; and FIG. 30B depicts a cross-sectional view of device 38d of FIGS. 13A-13G. As shown in FIG. 30A, magnetically attractive member 138a of device 38e includes a cylindrical member. In some embodiments, member 138a comprises a plurality of disc-shaped members, having substantially equal diameters, stacked to form a cylinder, as shown. In other embodiments, member 138a comprises a single-piece cylinder.

As shown in FIG. 30B, member 138a includes at least two sections each having a transverse dimension (e.g., diameter for circular cylinder, width for square or rectangular cylinder, etc.). In the embodiment shown, upper section 600 has a diameter 604, and lower section 608 has a diameter (or width) 612 that is larger than diameter (or width) 604 of upper section 600. For example, in some embodiments, diameter 612 can be equal to, larger than, less than, or between any of: 100, 110, 120, 130, 140, 150, 160, 170, 180, 190, and/or 200 percent of diameter 604. In some embodiments, upper section 600 comprises a plurality of disc-shaped members, having substantially equal diameters, stacked to form a cylinder, as shown. In some embodiments, upper section 600 comprises a single-piece cylinder. In some embodiments, lower section 608 comprises a plurality of disc-shaped members, having substantially equal diameters, stacked to form a cylinder, as shown. In some embodiments, lower section 608 comprises a single-piece cylinder. In some embodiments, upper section 600 and lower section 608 are unitary, such that member 138d comprises a single piece. Embodiments of the present medical devices having a lower section 608 that is larger than upper section 600 can be configured to have or contain a larger volume of magnetically attractive material (e.g., a member 138d with a larger volume than member 138a) so as to better maximize magnetic attraction between an external apparatus 34 and the device, while preserving and/or maintaining the structural integrity of the device (e.g., device 38d).

In the embodiment shown in FIG. 30A (and FIGS. 13A-13G), medical device 38d comprises a platform 166 having a proximal end, a distal end, and a length extending between the proximal end and the distal end; where platform 166 comprises a first magnetically-attractive member including an upper section 600 having a transverse dimension (e.g., diameter 604), and a lower section 604 having a transverse dimension (e.g., diameter 608) that is larger than the transverse dimension (e.g., diameter 604) of upper section 600. In the embodiment shown, platform 166 further comprises a second magnetically-attractive member 138d (e.g., that is substantially similar to the first magnetically-attractive member 138d). In the embodiment shown, each magnetically-attractive member 138d comprises a magnet (and/or a plurality of magnets). For example, in some embodiments, platform 166 has a coupling side 616; each magnet (and magnetically attractive member 138d) has an N pole and an S pole; and the N pole of one magnet (and magnetically attractive member 138d) is oriented toward coupling side 616, and the S pole of the other magnet (and magnetically attractive member 138d) is oriented toward coupling side 616. In the embodiment shown, upper portion 600 of each magnetically-attractive member is adjacent (and/or flush or even with) coupling side 616 of platform 166.

Although members 138a and 138d are each shown with a circular shape (e.g., circular cylinders), in other embodiments, members 138a and/or 138d can comprise square cylinders, rectangular cylinders, triangular cylinders, oval cylinders, and/or the like.

The various embodiments of the present systems, apparatuses, devices, and methods described in this disclosure can be employed and/or applied for any suitable medical or surgical procedures, including, for example, natural orifice transluminal endoscopic surgery (NOTES), single-incision laparosopic surgery (SILS), single-port laparoscopy (SLP), and others.

The various illustrative embodiments of systems, apparatuses, devices, and methods described herein are not intended to be limited to the particular forms disclosed. Rather, they include all modifications, equivalents, and alternatives falling within the scope of the claims. For example, although the version of cam slots 202 shown in platform 166 of device 38d extend all the way through the respective portions of the platform in which they reside, in other versions they can extend only partially into those platform portions such that they are not visible from either side of the platform.

The claims are not intended to include, and should not be interpreted to include, means-plus- or step-plus-function limitations, unless such a limitation is explicitly recited in a given claim using the phrase(s) “means for” or “step for,” respectively.

1.-45. (canceled)

46. A medical device comprising:
a platform at least partially defined by a length and a maximum transverse perimeter, the platform having a longitudinal recess that has a length defined along at least a portion of the length of the platform;
an arm having a proximal end, a distal end, and a length extending from the proximal end to the distal end, the arm coupled to the platform such that the arm is movable between (1) a collapsed position in which along the length of the recess the arm is disposed within the maximum transverse perimeter of the platform and (2) an expanded position in which the distal end of the arm is spaced apart from the platform; and
a cautery tool coupled to the arm.

47. The medical device of claim 46, where the platform comprises a magnetically-attractive material.

48. The medical device of claim 47, where the magnetically-attractive material includes a magnet.

49. The medical device of claim 48, where the magnetically-attractive material includes two magnets.

50. The medical device of claim 49, where:
the platform has a coupling side;
each magnet has an N pole and an S pole; and
the N pole of one magnet is oriented toward the coupling side, and the S pole of the other magnet is oriented toward the coupling side.

51. The medical device of claim 46, where the maximum transverse perimeter is less than about 7 inches.

52. The medical device of claim 51, where the area circumscribed by the maximum transverse perimeter is less than about 3.2 square inches.

53. A medical device comprising:
a platform at least partially defined by a length and a maximum transverse perimeter, the platform having a longitudinal recess that has a length defined along at least a portion of the length of the platform;
an arm having a proximal end, a distal end, a length extending from the proximal end to the distal end, and a longitudinal axis parallel to the length of the arm, the arm coupled to the platform such that the arm is movable between (1) an expanded position in which the distal end is spaced apart from the platform and (2) a collapsed position in which the distal end of the arm is closer to the platform than when the arm is in the expanded position; and
a cautery tool coupled to the arm and having a central axis parallel to the longitudinal axis of the arm;
where when the arm is in the collapsed position, the central axis of the cautery tool is disposed within the maximum transverse perimeter of the platform.

54. The medical device of claim 53, where the platform comprises a magnetically-attractive material.

55. The medical device of claim 54, where the magnetically-attractive material includes a magnet.

56. The medical device of claim 55, where the magnetically-attractive material includes two magnets.

57. The medical device of claim 56, where:
the platform has a coupling side;
each magnet has an N pole and an S pole; and
the N pole of one magnet is oriented toward the coupling side, and the S pole of the other magnet is oriented toward the coupling side.

58. The medical device of claim 53, where the maximum transverse perimeter is less than about 7 inches.

59. The medical device of claim 58, where the area circumscribed by the maximum transverse perimeter is less than about 3.2 square inches.

60. A medical device comprising:
a platform;
an arm coupled to the platform with a pin slidably disposed within a cam slot defined within one of the platform and the arm, the pin being coupled to the other of the platform and the arm, the arm movable between an expanded position and a collapsed position; and
a cautery tool coupled to the arm.

61. The medical device of claim 60, where the arm is coupled to the platform with two or more pins slidably disposed within first and second cam slots, the first and second cam slots defined within the platform, and the two or more pins supported by and in fixed relation to the arm.

62. The medical device of claim 61, where the platform comprises a magnetically-attractive material.

63. The medical device of claim 62, where the magnetically-attractive material includes a magnet.

64. The medical device of claim 63, where the magnetically-attractive material includes two magnets.

65. The medical device of claim 64, where:
the platform has a coupling side;
each magnet has an N pole and an S pole; and
the N pole of one magnet is oriented toward the coupling side, and the S pole of the other magnet is oriented toward the coupling side.

66.-81. (canceled)
